(19) United States
(12) Patent Application Publication
(10) Pub. No.: US 2002/0133132 A1
(43) Pub. Date: Sep. 19, 2002

(54) Absorbent article with a response surface

(76) Inventors: Marcelo S. Copat, Terre Haute, IN (US); Laurence T. Pearson, West Terre Haute, IN (US); Kellina Jo Simon, Brazil, IN (US)

Correspondence Address: JENKENS & GILCHRIST, PC, 1445 ROSS AVENUE, SUITE 3200, DALLAS, TX, 75202, US

(21) Appl. No.: 10/082,040

(22) Filed: Feb. 20, 2002

Related U.S. Application Data
(60) Provisional application No. 60/269,870, filed on Feb. 21, 2001.

(51) Int. Cl. B32B 3/10; A61F 13/15

(52) U.S. Cl. 604/383; 428/131

(57) ABSTRACT

A breathable absorbent article has at least one of a topsheet, an absorbent core, an optional distribution layer or a backsheet that comprises at least one to layer of a resilient, three dimensional liquid impervious polymeric film having apertures. The apertures form capillaries which are disposed at multiple directions, and/or have different diameters and lengths to create a response surface that is proportional to the pressure applied by the user's body, volume distribution of bodily fluid discharged by the user, the location of the discharge with respect to the position of the article, or a combination thereof, while the article is in use. The polymeric film can be formed by various processes, for example, vacuum forming or hydroforming methods.

This application claims the benefit of U.S. Provisional Application No. 60/269,870, filed Feb. 21, 2001.

### BACKGROUND OF THE INVENTION

1. Technical Field of the Invention

The present invention relates to absorbent articles, such as baby diapers, adult incontinent absorbent articles and in particular to sanitary napkins or panty liners. The articles usually include an absorbent core placed between a liquid pervious topsheet and a breathable backsheet. At least one of the layers included in at least one of the topsheet, the absorbent core or the backsheet comprises a resilient, three dimensional web which comprises a liquid management film, preferably polymeric film having apertures. The apertures form capillaries. The capillaries are disposed in multiple directions, and have multiple diameters, lengths and optionally other properties to create a response surface that is proportional to the pressure applied by the wearer's body while the article is in use, proportional to the volume distribution of bodily fluid discharged by the wearer, and proportional to the location of the discharge with respect to the position of the article. The invention is also directed to the apertured, three dimensional liquid management film which includes capillaries.

2. Description of Related Art

The main objectives of developments in the absorbent article field are to provide both a high level of protection, and a high comfort level to the wearer.

One means for providing consumer comfort benefits in absorbent articles is by the provision of breathable products. Breathability has typically concentrated on the incorporation of so called “breathable backsheets” in the absorbent articles. Commonly utilized breathable backsheets are microporous films and apertured formed films having directional fluid transfer as disclosed in for example, U.S. Pat. No. 4,591,523. Both of these types of breathable backsheets allow the evaporation of a portion of the fluid stored in the absorbent core and increase the circulation of air within the absorbent article. The air circulation is particularly beneficial as it reduces the sticky feeling experienced by many wearers during use, commonly associated with the presence of an apertured formed film or film like topsheet.

A well known problem associated with the use of breathable backsheets is that of liquid passage onto wearer's garments. Attempts to solve the problem have mainly resided in the use of multiple layer backsheets such as those illustrated in U.S. Pat. No. 4,591,523. Similarly European patent application Nos. EP 0 710 471 and EP 0 710 472 disclose breathable backsheets comprising layers of a gas permeable fibrous fabric and layers of apertured formed films having directional fluid transport. Such backsheets permit liquid passage when pressure is applied to the absorbent article (or the “pad”). The amount of pressure required to cause liquid passage is inversely proportional to the diameter of the capillaries. Because the passage of gasses is also proportional to the diameter of the capillaries, improved leakage protection reduces the breathability of the backsheet.

European patent application Nos. EP 0 934 735 and EP 0 934 736 improve upon EP 0 710 471 and EP 0 710 472 by incorporating in a backsheet of an absorbent article angled apertured formed film which has improved fluid management characteristics. Such films are also described in commonly assigned U.S. Pat. Nos. 5,562,932, 5,591,510, 5,718,928, and 5,897,543. In the films described in the above mentioned patents, the capillaries preferably are all substantially identical and homogeneously distributed across the film, therefore they exhibit a single elevation angle. Because the capillaries are disposed at an elevation angle (as defined herein) between 5 and 60 degrees or between 20 degrees and 85 degrees, the amount of pressure required to cause liquid passage is much greater than in the prior art films. However, the pressure closes the capillaries, therefore reducing the breathability of the backsheet while the pad is under pressure.

None of the above proposed developments have been able to provide a fully satisfactory solution to the problem of a breathable backsheet that allows minimum, if any, liquid passage under substantially all possible conditions. In particular, none of the above proposed solutions take into consideration the variation in pressure applied to the article while in use which results from variations in the contour of the wearer's body. Therefore, the prior art is limited by the competing requirements of breathability and liquid passage suppression.

Another means for providing consumer comfort benefits in absorbent articles (or “articles”) is by the provision of topsheets and distribution layers which acquire quickly bodily fluids from the body contacting surface of the pad, keep the fluids from returning to the body contacting surface when pressure is applied to the pad, and distribute the fluid evenly to the absorbent core so that core utilization is maximized. Core utility maximization enables reduction of core size which results in smaller, more comfortable pads. Rapid acquisition and retention has typically concentrated on the incorporation of so called “apertured formed film topsheets” in the absorbent articles. Commonly utilized apertured formed film topsheets are disclosed in for example, Thompson, U.S. Pat. No. 3,929,135, Mullane et al., U.S. Pat. No. 4,324,246, Radel et al., U.S. Pat. No. 4,342,314, and commonly assigned Raley et al., U.S. Pat. No. 4,252,516, Thomas et al., U.S. Pat. No. 4,535,020, and Junker et al., U.S. Pat. No. 5,591,510.

One well known problem associated with acquisition and distribution systems involving topsheets and distribution layers is the inability of these systems to maximize core absorption capacity and absorption rate, and consequently to minimize core size. Good fluid retention by the acquisition system depends on the ability of the core to quickly and permanently absorb the fluid transmitted to it by the acquisition system. A system that disperses acquired fluid quickly and evenly over the entire core surface enhances core absorption and capacity performance, therefore enabling construction of pads that utilize smaller cores. Attempts to solve the problem have mainly resided in the use of apertured formed film topsheets specialized for the acquisition and retention function, and distribution layers specialized for the distribution function. Such combinations fail to maximize core usage because each layer is specialized without consideration for the synergistic benefits that result from the design of a system comprised of acquisition and distribution layers which form a response surface that is proportional to the pressure applied by the wearer's body while the article is in use, proportional to the volume distribution of bodily fluid discharged by the wearer, and proportional to the location of the discharge with respect to the position of the article. In fact, the prior art has failed to suggest or disclose the design or provision of such a response surface.

It is therefore an objective of the present invention to provide an absorbent article, e.g., a disposable absorbent article, providing improved protection and comfort to the wearer and improved liquid passage suppression performance as compared to prior art articles.

### SUMMARY OF THE INVENTION

The invention relates to absorbent articles, such as disposable absorbent articles of a layered construction, such as baby diapers, adult incontinent articles, bandages and underarm sweat pads, and in particular sanitary napkins or panty liners. Typically, such articles comprise a liquid pervious topsheet forming the wearer facing surface of the article, an absorbent core (or “core”), and a breathable backsheet forming the garment facing (or contacting) surface of the article. The absorbent core is interposed between the topsheet and the backsheet. The absorbent core includes a liquid storage layer which comprises at least one absorbent material, such as hydrogel, superabsorbent, or a hydrocolloid material, in combination with suitable carriers. Such an article is referred to herein as an “absorbent article of a basic construction”.

The absorbent article may also include a distribution layer, which may be placed between the topsheet and the absorbent core or may be included in the absorbent core. The distribution layer is in fluid communication with the topsheet and the absorbent core, and it distributes liquid (acquired by the topsheet) to the absorbent core. Any one of the topsheet, the distribution layer, the absorbent core or the backsheet may be comprised of one or more layers of a suitable material.

The invention is also directed to an apertured film comprising a first surface and a second surface, which is spaced apart from the first surface. A plurality of apertured capillaries extend between the first and the second surface with each capillary being defined by a sidewall which extends from the first surface to the second surface. Each capillary has an elevation angle, and a surface angle.

In one embodiment, the capillaries in the film have multiple elevation angles. The remaining properties of the capillaries (such as the surface angle, surface area of one or both openings of the capillaries, length of the capillaries, surface texture of the walls of the sidewall of the capillaries, shape of the capillaries) may be substantially the same or different.

In another embodiment, the capillaries have multiple surface angles (or, are disposed at different surface angles). The remaining properties of the capillaries (such as the elevation angle, surface area of one or both openings of the capillaries, length of the capillaries, surface texture of the walls of the sidewall of the capillaries, shape of the capillaries) may be substantially the same or different.

In yet another embodiment, the capillaries have different (or a multiplicity of) water vapor permeabilities. The remaining properties of the capillaries (such as the surface and elevation angles, surface areas of one or both openings of the capillaries, length of the capillaries, surface texture of the walls of the sidewall of the capillaries, shape of the capillaries) may be substantially the same or different.

In a further embodiment, the capillaries have a multiplicity of levels of compression resistance. The remaining properties of the capillaries (such as the surface and elevation angles, water vapor permeabilities, surface areas of one or both openings of the capillaries, length of the capillaries, surface texture of the walls of the sidewall of the capillaries, shape of the capillaries) may be substantially the same or different.

In another embodiment, the film comprises at least a first region (or “type”) of apertured capillaries and a second region of apertured capillaries. Capillaries in the first region have a different elevation angle than the capillaries in the second region. The capillaries in the first region may have substantially the same as or different remaining properties than those of the second region. Such properties include surface area of one or both of the capillaries' openings, surface angle, shape and length of the capillaries, vapor permeability or surface texture of the walls of the capillaries' sidewall.

In one embodiment, the absorbent article of the basic construction, which may also comprise a distribution layer, includes in at least one of the topsheet, the backsheet, the absorbent core or the optional distribution layer any one of the films of the invention. In particular, the absorbent article includes an apertured film of the invention wherein the capillaries have multiple elevation angles, i.e., different capillaries having different elevation angles. The remaining properties of the capillaries may be substantially the same or different for all capillaries.

In another embodiment, the absorbent article of the basic construction, which may also comprise a distribution layer, includes in at least one of the topsheet, the backsheet, the absorbent core or the optional distribution layer any one of the films of the invention. In particular, the absorbent article includes an apertured film wherein the capillaries have multiple surface angles, i.e., different capillaries have different surface angles. The remaining properties of the capillaries may be substantially the same or different in all the capillaries.

In yet another embodiment, the absorbent article of the basic construction which may also comprise a distribution layer, includes in at least one of the topsheet, the backsheet, the absorbent core or the optional distribution layer any one of the films of the invention. In particular, the absorbent article includes an apertured film wherein the capillaries have different vapor permeabilities (or, a multiplicity of vapor permeabilities), i.e., different capillaries have different vapor permeabilities. The remaining properties of the capillaries may be substantially the same or different in all the capillaries.

In a further embodiment, the absorbent article of the basic construction, which may also comprise a distribution layer, includes in at least one of the topsheet, the absorbent core, or the optional distribution layer any one of the films of the invention. In a particular embodiment, the absorbent article includes an apertured film wherein the capillaries have different levels of compression resistance, i.e., different capillaries have different levels of compression resistance. The remaining properties of the capillaries may be substantially the same or different for all the capillaries.

In an alternative embodiment, the absorbent article of the basic construction, which may also comprise a distribution layer, includes in at least one of the topsheet, the backsheet, the absorbent core or the distribution layer an apertured film which comprises at least a first region (or type) of apertured capillaries and a second region of apertured capillaries. Capillaries in the first region have a different elevation angle than capillaries in the second region. The remaining properties of the capillaries of the first region may be substantially the same as or different than those of the capillaries of the second region.

In another embodiment of the invention, the absorbent article of the basic construction described above, which may also include a distribution layer, comprises at least one layer of an apertured film of the invention in at least one of the topsheet, the backsheet, the absorbent core or the optional distribution layer. The apertured film comprises at least a first region of apertured capillaries and a second region of apertured capillaries, with the capillaries in the first region having a different surface angle than the capillaries in the second region. The remaining properties of the capillaries of the first region may be substantially the same as or different than those of the capillaries of the second region.

Yet another embodiment of the invention is directed to an absorbent article of the basic construction described above, which may also include a distribution layer, wherein at least one of the topsheet, the backsheet, the absorbent core or the distribution layer includes at least one layer of the apertured film of the invention. The film used in this embodiment includes a first type of apertured capillaries having a different water vapor permeability than the second type of apertured capillaries. The remaining properties of the capillaries of the first type may be substantially the same as or different than those of the capillaries of the second type.

A further embodiment of the invention is directed to the absorbent article of the basic construction which also comprises a distribution layer. In this article, at least one of the topsheet, the backsheet, the absorbent core or the distribution layer includes at least one layer of the apertured film of the invention. The film used in this embodiment includes capillaries of the first region which have a different level of compression resistance than capillaries of the second region. The remaining properties of the capillaries of the first region may be substantially the same as or different than those of the capillaries of the second region.

In another embodiment, the film comprises multiple regions,(or “types”) of apertured capillaries. Capillaries in each region are arranged in a regulated pattern, unique to that region, and different than regulated pattern(s) of capillaries in the other regions.

Films of the invention can be made from a suitable polymer, such as polyethylene, e.g., low density polyethylene (LDPE) or linear low density polyethylene (LLDPE) or a combination (blend) thereof.

Additionally, we discovered that films of the invention and any apertured films known heretofore of a similar construction (i.e. prior art films) can be improved by an embodiment of the invention. Such heretofore known films comprise a plurality of apertured capillaries extending between a first surface and a second surface of the film, with each capillary being defined by a sidewall which extends from the first to the second surface. Each capillary has at least an elevation angle and may have a surface angle. Such heretofore-known films have also usually been made from LDPE, LLDPE or a combination thereof. Such prior art films include film embodiments having a multiplicity of capillaries with all capillaries being substantially identical and homogeneously distributed across the film, e.g., see Carlucci, et al., EP 0 934 735, i.e., all such capillaries have substantially the same elevation angles and substantially the same surface angles. When the films (of the invention or the prior art films) are made, they are wound on a roll. When they are unwound, their liquid permeability usually decreases. Also, the structures of the capillaries of the unwound films are uneven, e.g., the ends of the capillaries are often uneven. We discovered that using a mixture of at least about 10%-by weight, or about 10% to about 50% by weight, of high density polyethylene (HDPE) or medium density polyethylene (MDPE) and the remainder LDPE, LLDPE or a blend of LDPE and LLDPE improves the resistance of the resulting film (i.e. either the film(s) of the invention or the prior art film(s)) to leakage under pressure (and improves the film's resiliency or compression resistance). This also improves the structures of the capillaries, i.e., the ends of the capillaries are substantially even. The resistance to leakage under pressure of the resulting films is substantially the same when the films are wound into a roll or unwound.

In at least some embodiments, the capillaries are arranged in such a manner as to attain objectives of the invention, i.e., an absorbent article wherein at least one of its components (such as the topsheet, the backsheet, the absorbent core or the optional distribution layer) has a response surface and the desired vapor permeability and fluid (or liquid) management properties.

### BRIEF DESCRIPTION OF THE DRAWINGS

FIG. 1 shows a cross-sectional view of a prior art apertured film comprising capillaries with a single elevation angle and a single surface angle.

FIG. 2 shows a cross-sectional view of an angled apertured film of this invention comprising capillaries in two different regions with capillaries in a first region having different surface angles than in the second region.

FIG. 3 shows a cross-sectional view of an angled apertured film of the invention comprising angled capillaries oriented at multiple surface angles and also multiple elevation angles.

FIG. 4 shows a cross-sectional view of an angled apertured film of the invention comprising angled capillaries oriented at multiple surface angles and multiple elevation angles which also have a multitude of lengths.

FIG. 5 shows a schematic top view of a backsheet of the invention illustrating areas of the article having a 90 degree surface angle.

FIG. 6A shows a schematic top view of an absorbent article of the invention with a backsheet of the invention including an apertured film utilizing capillaries having four surface angles, A, B, C and D.

FIG. 6B shows a cross-sectional view of the backsheet of FIG. 6A with capillaries exposed.

FIG. 7 shows a schematic top view of a backsheet of an absorbent article of the invention comprising angled capillaries, arranged into eight types, each type having a different surface angle, surrounding an area R of maximum fluid discharge. The area R may also include capillaries, thereby adding a ninth type of capillaries. The capillaries in the area R may have a surface elevation angle of 0 degrees and open area of zero.

FIG. 8 shows schematic top view of a film of the invention.

FIG. 9 is a schematic top view of a backsheet of an absorbent article of the invention comprising a film of the invention with angled capillaries, arranged into two regions, capillaries in each region having a different direction and a different surface angle. Symbol R designates the area of maximum fluid discharge, which may also include capillaries. The capillaries in the area R may have surface and elevation angles of 0 degrees and open area of zero.

FIG. 10 is a schematic top view of a backsheet of an absorbent article of the invention comprising a film of the invention with angled capillaries, arranged into four regions, the capillaries in each region having a different direction and a different surface angle. Symbol R designates the area of maximum fluid discharge, which may also include capillaries, thereby forming a fifth region. The capillaries in the region R may have surface and elevation angles of 0 degrees and open area of zero.

FIG. 11 is a side view of one embodiment of a capillary used a film of the invention.

FIG. 12 is a side view of another embodiment of a capillary used in a film of the invention.

FIG. 13 is a side view of another embodiment of a capillary used in a film of the invention.

FIG. 14 is a side view of yet another embodiment of a capillary used in a film of the invention.

FIG. 15 is a side view of an alternative embodiment of a capillary used in a film of the invention.

FIG. 16 is side view of another embodiment of a capillary used in a film of the invention.

FIG. 17 is a perspective view of an exemplary screen for use in constructing a film of the invention.

FIG. 18 is a cross-sectional view of another exemplary screen for use in constructing a film of the invention.

FIG. 19 is a schematic perspective view of an exemplary apparatus shown forming a film having lanes of a first regulated pattern near the edges and a second regulated patter in the center in accordance the invention.

FIG. 20 is a schematic perspective view of an exemplary apparatus shown forming a film having lanes of a first regulated pattern near the edges and a combined first and second regulated patter in the center in accordance the invention.

FIG. 21 is a cross-sectional detail view of another exemplary screen for use in constructing a film of the invention.

### DETAILED DESCRIPTION OF THE EXEMPLARY EMBODIMENTS OF THE INVENTION

In general, the topsheet should have good liquid acquisition and rewet prevention to maintain a dry surface and thereby keep the skin of the wearer dry; the absorbent core needs to provide enough absorbent capacity and allow the flow of vapor and/or air through it; and the backsheet should prevent liquid passage while being sufficiently breathable. Furthermore, the individual elements of the absorbent article (i.e. the topsheet, the optional distribution layer, the absorbent core and the backsheet) are joined, using any techniques known in the art so that the final article has the desired comfort and performance level.

Set forth below are definitions of some terms used herein.

The term “substantially” means that a given property or parameter (such as the surface angle) may vary by about 30% from the stated value.

As used herein, the term “areas immediately surrounding the region of maximum fluid discharge” means a surface area surrounding the region of maximum fluid (i.e. liquid) and/or solid waste discharge and extending approximately 1 inch in all directions from that region. The term “periphery” “peripheral areas” or “areas peripheral to” means the surface area other than the area of maximum fluid discharge and the areas immediately surrounding it.

The term “multiplicity” as used in conjunction with various parameters and properties of the apertured capillaries such as elevation angle, surface angle, and permeability, means that individual capillaries have a particular value for each parameter or property (rather than multiple values of each parameter in a single capillary), but different capillaries with different respective parameters or properties can be placed in a given area. Of course, as discussed herein, multiple capillaries having substantially the same value of a given parameter or property may be grouped together in a given area (or region).

The term “regulated pattern” means regions of capillaries in the film (and therefore in the component(s) of the absorbent article which include the film) where a selected property or selected properties of the capillaries are repeatably controlled, i.e., the property or properties is or are controlled to achieve a desired pattern of the selected property or properties. If a region comprises a regulated pattern of capillaries, it does not necessarily mean that all capillaries in that region have exactly the same property (or properties) which was selected to be controlled. It means that the selected property is varied in a designed, prescribed manner (or pattern) to substantially achieve a particular formula. Each region has only one regulated pattern. For example, two regions where the capillaries in both regions have the same properties, except elevation angle, are two regions (or zones) each having a unique regulated pattern which is different from the regulated pattern in the other zone. The properties which can be selected to be controlled include: elevation angle, surface angle, vapor permeability, liquid permeability, compression resistance, surface areas of one or both openings of the capillaries, length of the capillaries, texture of the walls of the sidewall of the capillaries and shape of capillaries.

The term “permeability” refers to the permeability of vapor or liquid.

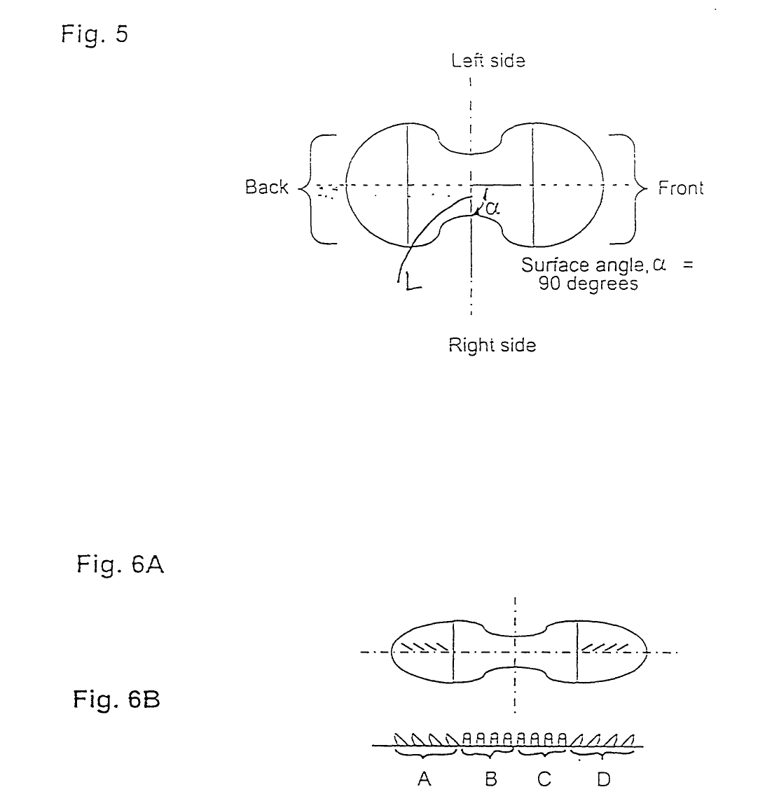

The direction of a capillary is defined by the direction of the capillary centerline which is defined by an elevation angle and a surface angle. The elevation angle is the angle between the capillary centerline and a plane parallel to a 1st surface of the film (or “web”) containing the capillary. The surface angle is the angle measured in a counter clock-wise direction, between the vertical projection of the capillary centerline onto the plane of the 1st surface of the film and a base line. The “base line” is a line drawn on the 1st surface of the film, which line is parallel to the transverse direction, intersects the initial point of the vertical projection and extends towards the right side of the article. (An example of base line is designated by “L” in FIG. 5.) The front side of the article is the area located at the longitudinal extreme in a direction defined by a surface angle of 0 to 180 degrees. The back side of the article is the area located at the longitudinal extreme in a direction defined by a surface angle of 180 to 360 degrees. See FIG. 5.

In at least some embodiments of the films of this invention, the elevation angle of the capillaries ranges from about 0 to about 90 degrees, preferably it is at least about 5 degrees. In some embodiments, the elevation angle may range from about 5 to about 80 degrees, or from about 5 to about 70 degrees.

The article has a length and a width defined by two perpendicular centerlines (or lines) that intersect substantially at the center of the article. The length is defined by the length of the longest of the two intersecting lines, and the width is defined by the shortest of the two intersecting lines. The longitudinal direction is the direction parallel to the length of the article, and the transverse direction is the direction perpendicular to the longitudinal direction. The left side is defined as the area of the article located on the left (north) side of the longitudinal centerline when the backsheet of the article is face up, i.e., the area defined by a surface angle between 90 and 270 degrees. The right side is defined as the area of the article located opposite the left side, i.e., the area defined by surface angles of 0 to 90 and 270 to 360 degrees. See FIG. 5.

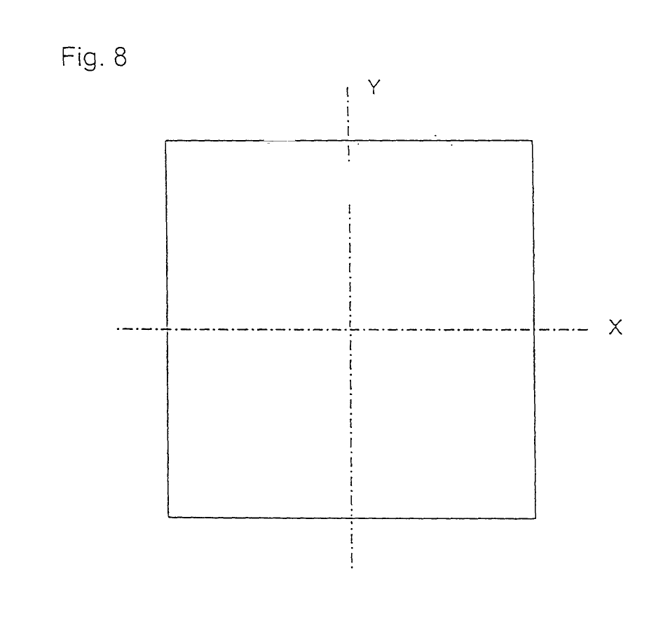

Similarly, the film of the invention has a length and a width defined by two perpendicular centerlines (or lines) that intersect substantially at the center of the film. The length is defined by the length of the longest of the two intersecting lines, and the width is defined by the shortest of the two intersecting lines. If the film has the shape of a square, the length is defined by the length of the line extending along the “X” axis and the width by the line extending along the “Y” axis (see FIG. 8). The longitudinal direction is the direction parallel to the length of the film and the transverse direction is the direction perpendicular to the longitudinal direction. The left side is defined as the area of the film located on the left (north) side of the longitudinal centerline when the film is face up, i.e., the area defined by surface angles between 90 and 270 degrees. The right side is defined as the area of the film located opposite the left side, i.e., the area defined by surface angles of 0 to 90 and 270 to 360 degrees.

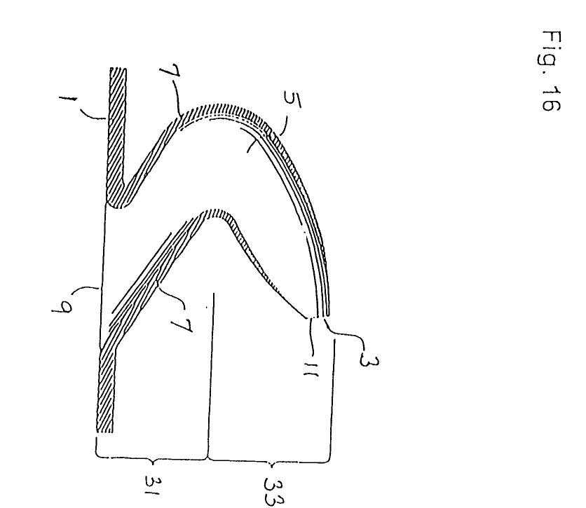

If a capillary includes two portions, oriented in different directions (e.g. see FIG. 16), the capillary centerline of the portion comprising the 2nd surface opening of the capillary (e.g. portion 33 in FIG. 16) is used to define the direction of the capillary. As discussed herein, the film of the invention can be included in one or more of the following components of the absorbent article: the topsheet, the optional distribution layer, the absorbent core or the backsheet.

For purposes of illustration only, the invention will now be described in conjunction with the inclusion of the film of the invention in a breathable backsheet of the absorbent article of the invention. Nonetheless, all the principles and embodiments of the utilization of the film of the invention in the backsheet are equally applicable to the utilization of the film in any other components of the absorbent article, i.e., the topsheet, the distribution layer or the absorbent core.

In one embodiment, the breathable backsheet comprises at least one response surface breathable layer (or web) which comprises a resilient three dimensional film of the invention with apertured capillaries disposed at multiple directions (i.e. multiple elevation and surface angles) and/or having different diameters and/or lengths.

The response surface web has a 1st surface comprising multiple 1st capillary openings and a 2nd surface comprising multiple 2nd capillary openings. Each capillary originates in the 1st surface of the web and extends away from it. At the end of each capillary is a 2nd capillary opening which is connected to the 1st capillary opening by the capillary wall. If the web (or the film) is placed in the topsheet, the 1st surface is that surface of the web (or the topsheet) which contacts the wearer and the second surface faces away from the wearer. If the web (or the film) is placed in a backsheet, the first surface may be facing away from the wearer. Those skilled in the art would readily understand how to place the web (or the film) in the distribution layer or absorbent core.

The capillaries may be arranged in regions, with each region having a regulated pattern of capillaries. Without wishing to be bound by any operability theory, it is believed that such an arrangement of capillaries improves the balance of the breathability properties and liquid passage suppression properties of the backsheet and enables a better match between the shape of the wearer's body and its fluid discharge location with the type of each capillary, its location and three-dimensional shape of each capillary. Again without being bound by an operability theory, such an arrangement of capillaries is believed to enable parts of a user's body which come into contact with the film to seek its own custom “fit” among the capillaries best suited to the shape of the body part.

In one embodiment, capillaries have different 1st capillary opening diameters, such that the permeability of the web in the area of maximum fluid discharge is reduced and the permeability in the peripheral areas of the web (i.e. away from the area of maximum fluid discharge) is increased, therefore maintaining substantially constant average vapor and liquid permeability of the backsheet, while further improving fluid passage suppression in the area of maximum fluid discharge. This can be accomplished, for example, by providing capillaries with smaller 1st capillary opening diameters in the area of maximum fluid discharge (and areas immediately surrounding it) and with larger 1st capillary opening diameters in the peripheral areas of the web.

In yet another embodiment, capillaries have different lengths, such that the liquid permeability of the web in the area of maximum fluid discharge is reduced and the liquid permeability in the peripheral areas of the web is increased, therefore maintaining average liquid permeability of the backsheet while further improving fluid passage suppression in the area of maximum fluid discharge. This can be done, for example, by providing capillaries with greater length in the area of maximum fluid discharge (and areas immediately surrounding it) than in the peripheral areas of the web.

As is known to those skilled in the art, every absorbent article, including disposable absorbent articles, has a region of maximum fluid discharge which may be different in different articles. For example, such a region of maximum fluid discharge is different in an adult incontinent article than in a feminine sanitary napkin. In general, the absorbent article of the invention incorporates the film of the invention in such a manner that average liquid permeability is minimized across substantially the entire surface of the article, while average vapor (particularly water vapor) permeability is maximized across substantially the entire surface. Applicants have discovered that by adjusting properties of the capillaries of the film and placing the capillaries in particular locations on the backsheet, properties of the backsheet (and therefore the absorbent article) can be controlled. For example, the backsheet of the invention may have substantially the same liquid suppression characteristics and better vapor permeability, substantially improved liquid suppression characteristics and substantially the same vapor permeability, or improved liquid suppression characteristics and improved vapor permeability than backsheets of the prior art. These properties can be controlled by adjusting the vapor permeability and liquid permeability of the backsheet in the region of maximum fluid discharge and areas immediately surrounding it, on the one hand, and in the areas of the backsheet peripheral to the region of the maximum fluid discharge (and, optionally, areas immediately surrounding it), on the other hand.

For example, the backsheet of the invention, having substantially the same liquid suppression properties (i.e. substantially the same liquid permeability) but better vapor permeability properties than a backsheet of prior art can be obtained by increasing the vapor permeability in the periphery of the backsheet while maintaining these properties in the region of maximum fluid discharge and areas immediately surrounding it substantially the same. The backsheet of the invention having substantially improved liquid suppression properties (i.e. lower liquid permeability), yet substantially the same vapor permeability as a backsheet of prior art, may be obtained by decreasing the vapor permeability in the region of maximum fluid discharge and areas immediately surrounding it, and increasing it in the periphery of the backsheet. The backsheet of the invention having improved liquid suppression properties (lower liquid permeability) and better vapor permeability may be obtained by reducing liquid permeability in the region of maximum fluid discharge and areas immediately surrounding it, and substantially increasing it in the periphery of the backsheet.

Properties of the capillaries and their placement are controlled to achieve these objectives. Since the periphery of the backsheet usually has a substantially lower accumulation of liquid than the region of maximum fluid discharge and areas immediately surrounding it, the vapor permeability is increased substantially more than its liquid passage (i.e. liquid permeability) when the vapor permeability and liquid permeability of the film is increased in the peripheral area. Conversely, the region of maximum fluid discharge and the areas immediately surrounding it usually have a greater accumulation of liquid than in the peripheral area. Thus, decreasing the vapor permeability and liquid permeability of the film in the region of maximum fluid discharge and the areas immediately surrounding it has a significant effect on decreasing liquid permeability of the backsheet and the negative effects of reduced vapor permeability in the region of maximum fluid discharge can be compensated by increasing it in the peripheral areas.

Vapor permeability and liquid permeability can be adjusted in various ways. For example, adjusting elevation angle capillaries changes liquid permeability and may change vapor permeability of the capillaries. Adjustment of elevation angle of capillaries may affect liquid permeability in a different fashion than vapor permeability. Increasing the 1st surface capillary opening increases both vapor and liquid permeability. Similarly, directing the apertured capillaries away from the area of maximum liquid discharge reduces their liquid permeability.

The main advantage that capillaries have over two dimensional holes is their ability to apply capillary forces to the liquid, therefore drawing the liquid into the 1st opening of the capillary and causing it to exit through the 2nd opening. Capillary suction performance is highly influenced by the liquid's chemical composition. For example, capillary performance on menses is different from capillary performance on urine. Some of the known performance levers are surface energy, wall texture, capillary shape, capillary length, material composition, 1st opening diameter, 2nd opening diameter, etc. It is known, for example, that the minimum diameter 1st opening diameters (for circular shaped 1st openings) must be larger in articles focused on menses management than in articles focused on urine management, primarily due to the lower viscosity of urine. It is therefore not possible to describe in absolute terms all of the combinations of capillary parameters that result in a particular benefit without as well describing the fluid, the article design, and the placement of the film in the article. A person skilled in the art will understand how to select capillary parameters and regulated patterns to fit the specific design criteria of the article, including knowledge of the other required elements of the article such as the core and bonding elements (e.g. adhesives, bonding methods).

At least some embodiments of the film and absorbent articles of the invention are intended to create a response surface which is formed in response to and proportional to pressure imparted on the film by the wearer's body, in response to and proportional to the volume distribution of bodily fluid discharged by the user, in response to and proportional to the location of the discharge with respect to the position of the article, or a combination thereof, when the film is incorporated into an absorbent article and the article is in use. The response surface may resemble a typographical map of a portion of a terrain which may include elevations and valleys.

Thus, the invention provides a means of custom designing the film in such a fashion that, when it is in use in an absorbent article, the aforementioned three factors may combine in a variety of ways to create a response surface which is proportional to one or more of the factors. The effect of the factors may vary, depending on the use of the absorbent article and other conditions. For example, pressure points in a feminine hygiene article are different than in a child's diaper, pressure exerted by solid waste is different than that exerted by liquid waste.

In all embodiments of the invention, the area of the maximum fluid discharge in the backsheet and areas immediately surrounding it may be free of capillaries, in which case that region of the backsheet will be impermeable to fluid and vapor. Alternatively, the area of maximum fluid discharge and areas immediately surrounding it may include capillaries of the invention which can be designed by persons skilled in the art to impart to those areas desired vapor permeability and liquid permeability suppression characteristics in accordance with this invention.

In one embodiment, two or more regions of regulated patterns of capillaries are used. In the first region, having a first regulated pattern of capillaries, all individual capillaries have substantially the same elevation angles or all capillaries may have different elevation angles. Similarly, in the second (or any further) region having a second (or any further) regulated pattern of capillaries, all individual capillaries have substantially the same elevation angle or all capillaries may have different elevation angles. Thus, it is possible, in the first region to have individual capillaries having substantially the same elevation angle as one or more individual capillaries in the second region, so long as the film, when incorporated into the backsheet, is capable of forming at least one response surface as described above when the article is in use, or the film can be used to impart to the backsheet the desired fluid permeability and vapor permeability characteristics.

In one embodiment, the film includes a plurality of apertured capillaries extending between a first surface and a second surface of the film, with each capillary having an elevation angle and a surface angle. The film comprises at least a first region of regulated pattern and a second region of regulated pattern of apertured capillaries, with the first region including apertured capillaries having a different elevation angle than the second region of apertured capillaries. In one embodiment, each capillary in the first region of capillaries has an elevation angle which is different from the elevation angle of any capillary in the second region of capillaries. In the first region, all individual capillaries may have substantially the same surface angle (or all individual capillaries may have different surface angles). Optionally, in the first region, all individual capillaries may have substantially the same elevation angle (or they may have different elevation angles). Also, in the second region, all individual capillaries may have substantially the same surface angle (or they may have different surface angles). Similarly, in the second region, all individual capillaries may have substantially the same elevation angles or they may have different elevation angles.

Other parameters (or properties) of individual capillaries within the first region and the second region may also be adjusted to obtain a film forming at least one of the aforementioned response surfaces. The first region of regulated pattern of capillaries may have the following additional properties (or parameters), any one of which may be different from, or may be substantially the same as, the respective properties of the second region of regulated pattern of capillaries: surface area of the first and second capillary opening; the shape or diameter of the capillary; the length of the capillary; surface texture of the interior wall of the capillary. In one embodiment, each of the aforementioned parameters in substantially all capillaries of the first region is substantially the same. In another embodiment, each of the aforementioned parameters in substantially all capillaries of the second region of capillaries is substantially the same.

Turning now to the drawings, FIG. 1 illustrates a cross-section of a prior art film where all capillaries have the same elevation angle and the same surface angle.

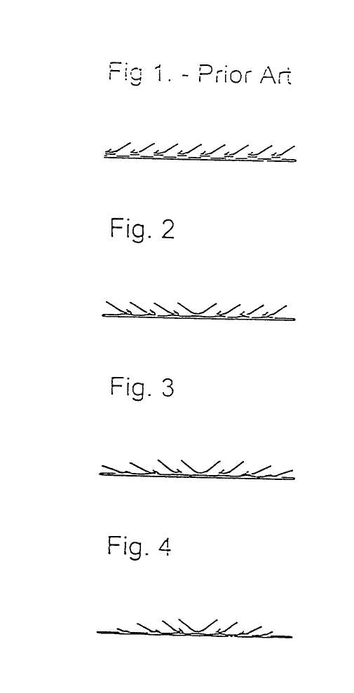

FIG. 2 illustrates a cross-section of one embodiment of a film of this invention where the capillaries are arranged in two different regions. In a first region, all the capillaries have substantially the same surface angle and in a second region all the capillaries have substantially the same surface angle, which is different from that of the capillaries in the first region. All the other properties of the capillaries in the first region are substantially the same as the respective properties of the capillaries in the second region. Thus, the first region includes one regulated pattern of capillaries, while the second region includes a different regulated pattern of capillaries.

FIG. 3 shows a cross-section of a film of another embodiment of the invention, where the capillaries are also arranged into two different regions. A first region includes a first regulated pattern and a second-region a second regulated pattern of capillaries. In the first regulated pattern, all capillaries have substantially the same surface angle and elevation angles. In the second regulated pattern all capillaries have substantially the same surface and elevation angles, which are different from those of the capillaries of the first regulated pattern. All the other properties of the capillaries in the first regulated pattern are substantially the same as those of the capillaries of the second regulated pattern.

FIG. 4 shows a cross-section of a film of yet another embodiment of the invention where the capillaries are also arranged into two different regions. A first region includes a first regulated pattern and a second region a second regulated pattern of capillaries. In the first regulated pattern all capillaries have substantially the same surface angle and elevation angle and substantially the same length. In the second regulated pattern all capillaries have substantially the same surface and elevation angles, and substantially the same lengths. The surface and elevation angles and the lengths of the capillaries in the second regulated pattern are different from those of the first regulated pattern. All the other properties of the capillaries in the first regulated pattern are substantially the same as those of the capillaries in the second regulated pattern.

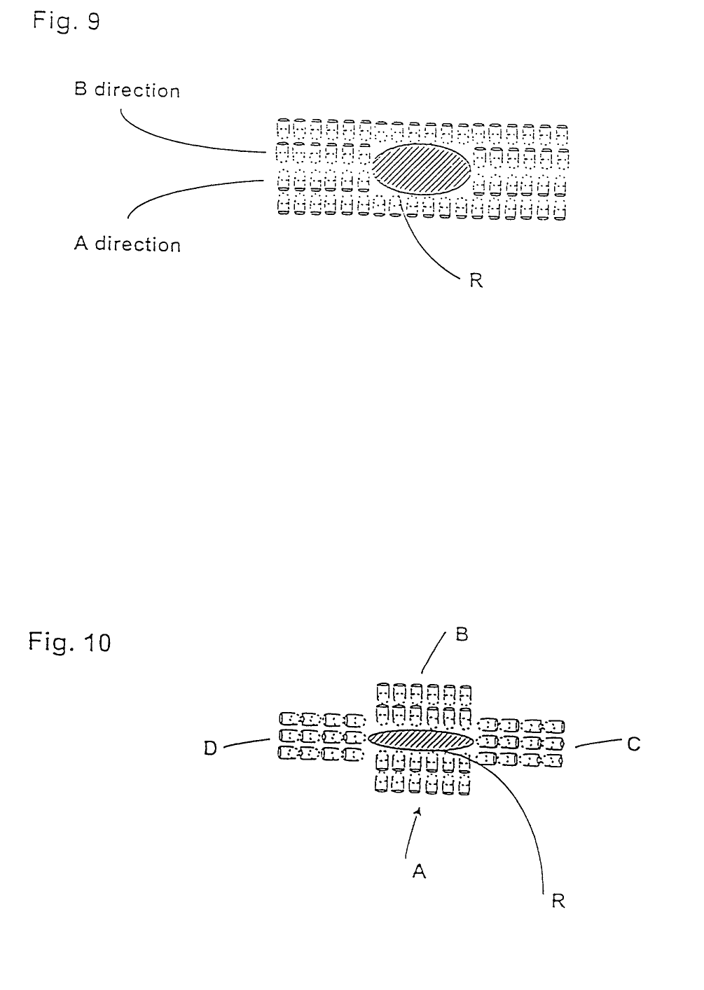

In another embodiment, illustrated in FIG. 9, the backsheet comprises capillaries disposed in two directions, A and B (i.e. two regions of regulated pattern of capillaries, a first region having a first regulated pattern and a second region having a second regulated pattern). Direction A is the direction defined by a surface angle of 0 degrees and an elevation angle of 45 to 70 degrees. Direction B is the direction defined by a surface angle of 180 degrees and an elevation angle of 45 to 70 degrees. All the capillaries on the right side of the web are disposed at the A direction and all the capillaries on the left side of the web are disposed at the B direction. All the other properties of the capillaries in the first region are substantially the same as in the second region. Without being bound by any operability theory, it is believed that by aligning the angles of the two regions of capillaries in opposite directions it is possible to deflect fluid from the center region R of the article (which is the region of maximum fluid discharge), thereby increasing fluid passage suppression without substantially affecting (i.e. decreasing) breathability (i.e. vapor permeability) of the backsheet. The center region R may also comprise capillaries disposed at the A and B directions or it may be comprised of a solid, liquid impermeable film.

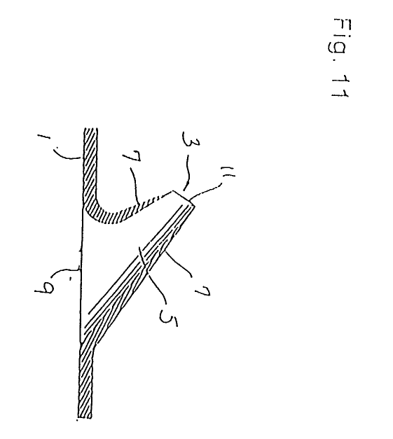

In another embodiment, capillaries are disposed in four directions A, B, C and D (i.e. four regions of capillaries each region including a different regulated pattern, FIG. 10). Directions A and B (first and second regulated pattern, respectively) are the same as in the embodiment of FIG. 9. Direction C (third regulated pattern) is the direction defined by a surface angle of 90 degrees and an elevation angle of 45 to 70 degrees. Direction D (fourth regulated pattern) is the direction defined by a surface angle of 270 degrees and an elevation angle of 45 to 70 degrees. Capillaries around the center region R of the article are disposed at the A direction on the right side and the B direction on the left side, and capillaries in front and back sides of the web are disposed at the C and D directions, respectively. Without being bound by any operability theory, it is believed that by aligning the capillary angles in all directions away from the area of maximum fluid discharge R it is possible to further increase fluid passage suppression without substantially affecting (i.e. decreasing) breathability of the backsheet. The center region R may also comprise capillaries disposed at the A, B, C and D directions (so they substantially follow the pattern of the capillaries around the center region R). This is illustrated in FIGS. 6A and 6B, where the capillaries are arranged in four different regulated patterns, each regulated pattern including capillaries having directions A, B, C, D, respectively, which are the same as in the embodiment of FIG. 10. All the other properties of the capillaries in the individual regulated patterns of FIGS. 10 and 6A, 6B are substantially the same as in the other regulated patterns of the respective Figures. Alternatively the center region R may be comprised of a solid, liquid impermeable film.

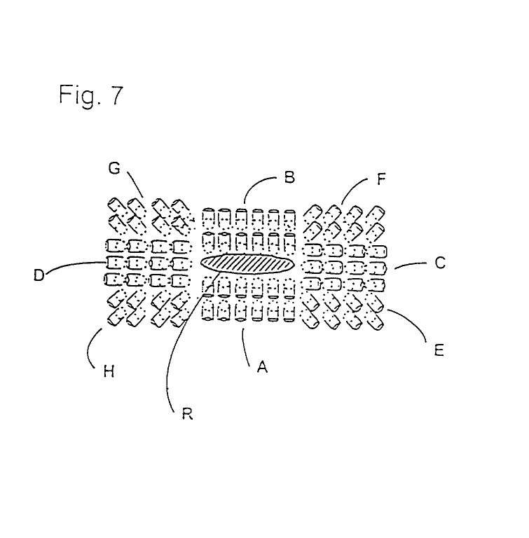

FIG. 7 shows an embodiment of the invention utilizing eight different regions of apertured capillaries, with capillaries in each region having different surface angles. Thus, each of the eight regions includes one distinct regulated pattern of capillaries. The eight regions surround the area of maximum liquid discharge R, and capillaries in those eight regions are disposed in eight directions, A, B, C, D, E, F, G, H and 1. Directions A, B, C and D are the same as in the embodiment of FIG. 10, direction E is defined by a surface angle of approximately 45 degrees and an elevation angle of 45 to 70 degrees, direction F by a surface angle of approximately 135 degrees and elevation angle of 45 to 70 degrees, direction G by a surface angle of approximately 225 degrees and elevation angle of 45 to 70 degrees and direction H by a surface angle of approximately 315 degrees and elevation angle of 45 to 70 degrees. All the capillaries in each region have substantially the same elevation and surface angles. All the remaining properties of the capillaries of regulated pattern are substantially the same as the respective properties of the other regulated patterns of this embodiment.

Various shapes, sizes and configurations of the capillaries are possible. Some of the possible shapes are illustrated in FIGS. 11-16. The apertures 3 form capillaries 5 which have side walls 7. The capillaries extend away from the first surface of the film 1 for a length which typically should be at least in the order of magnitude of the largest diameter of the aperture. The capillaries have a first opening 9 in the plane of the 1st surface of the film 1 and a second opening 11 which is the opening formed when the suction force (such as a vacuum) in a vacuum-forming process creates the aperture. The edge of the second opening may be rugged or uneven, comprising loose elements extending from the edge of the opening. However, it is preferred that the opening be as smooth as possible so as not to create a liquid transport entanglement between the extending elements at the end of the second opening 11 of the capillary 5 with an absorbent core in the absorbent article (in contrast this may be desirable for apertured film topsheets where such loose elements provide the function of sucker feet).

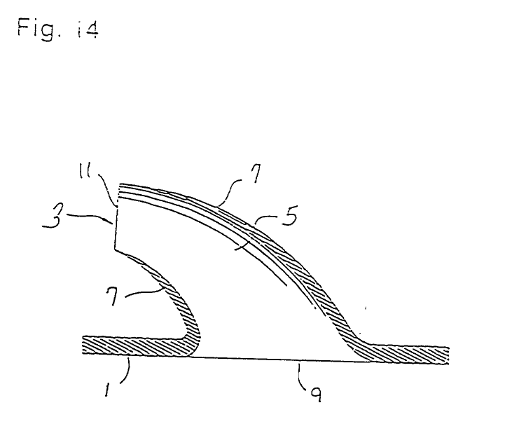

As shown in FIG. 13 the first opening has a center point 19 and the second opening also has a center point 111. These center points for noncircular openings are the area center points of the respective opening area. When connecting the center point 19 of the first opening 9 with the center point 111 of the second opening 11 a capillary centerline 13 is defined. This capillary centerline 13 forms an angle p with the plane of the film which is the same plane as the garment facing surface 1 of the film, if the film is included in the backsheet. (If the film is included in the topsheet, the first surface 1 may face towards the wearer).

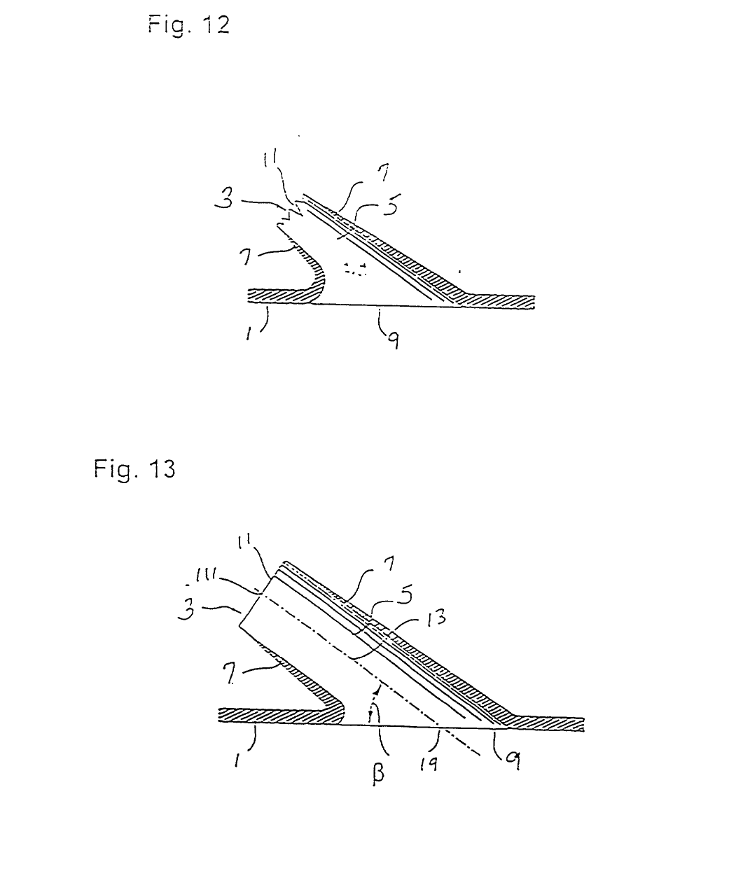

It is of course possible to allow the capillaries to take the shape of a funnel such that the second opening 11 is substantially smaller than the first opening 19 when considering the opening in a plane perpendicular to the center axis 13. Such an embodiment is shown in FIGS. 11 and 12. In FIG. 11 it is also shown that the wall 7 of the capillary may not end in a second opening 11, such that the opening forms a surface perpendicular to the center axis 13 but such that the wall on the portion of the capillary further apart from the 1st surface of the film 1 extends over the opening to further aid the film in reducing the probability of liquid entering the capillaries through the 2nd opening of the film and traveling to the 1st surface of the film (and cause leakage).

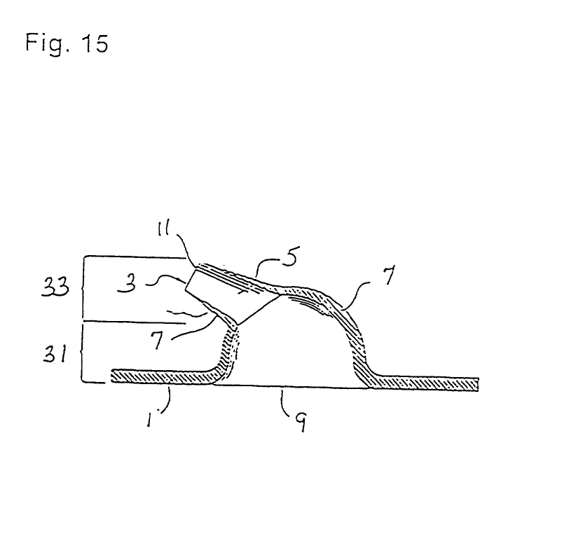

In FIG. 14 another embodiment of the capillaries useful for the present invention is shown which is curved along its length towards the second surface of the film, and away from the first surface 1. This has a similar effect as the extension of the wall 7, as shown in FIG. 15.

In FIG. 15, an embodiment of a capillary of the invention is shown which has a first portion 31 and a second portion 33. The first portion 31 is oriented in a different direction than the second portion 33 of the capillary. The difference can also be in shape, size and form of the portions of the capillaries to achieve the desired level of breathability (i.e. vapor permeability), while suppressing liquid permeability of the film in the direction from the wearer facing side towards the garment facing side (i.e. from the second to the first surface in FIG. 15). The capillary may also have a funnel shape oriented so that the narrower dimension is in the wearer-facing surface and the wider dimension in the garment-facing surface as described, for example, in Depner et al., European published patent application EPO 710 471 AI, the entire contents of which are incorporated herein by reference in the manner consistent with this disclosure and the invention. Capillaries may have a relatively narrow first opening (such as a diameter if they have a circular cross section) and relatively wider second opening.

In FIG. 16 another preferred embodiment of a capillary according to the present invention is shown which has a first portion 31 and a second portion 33. The first portion 31 of the capillary is oriented in a different direction than the second portion 33 of the capillary 5. The two portions may also have different shape, size, and form of the portions of the capillary in order to achieve the desired level of breathability while preventing liquid passage through the film in a direction from the wearer facing side (from the second surface) towards the garment facing side (the first surface). Such an example is shown in FIG. 15.

The capillaries may have any suitable cross-section such as circular, oval, rectangular, or square.

Film Making Methods

The film of this invention can be made by any method known in the art, e.g., by vacuum forming or hydroforming methods.

One of the earlier methods for vacuum perforation of a thermoplastic film is disclosed in Zimmerli, U.S. Pat. No. 3,054,148, incorporated herein by reference in the manner consistent with this invention and application. The patentee describes a stationary drum having a molding element or screen (collectively “screen”) mounted around the outer surface of the drum and adapted to freely rotate thereon. A plasticized thermoplastic material is applied onto the screen. A vacuum chamber is employed beneath the screen to create a pressure differential between the respective surfaces of the thermoplastic sheet to be perforated to cause the plasticized sheet material to flow into openings provided in the screen and thereby cause a series of openings, holes or perforations to be formed in the plastic sheet or film. Zimmerli discloses a method for making a film with tapered capillaries. A variety of methods and apparatuses including different types of perforating screens or rotatable molding elements have been developed over the years since Zimmerli's invention for making apertured polymer films. Examples of such methods and apparatuses are described in U.S. Pat. Nos. 4,155,693, 4,252,516, 4,508,256, and 4,509,908, incorporated herein by reference in the manner consistent with this application and the invention described herein.

The film of the invention may also be produced by a hydroforming process described in detail in Curro, U.S. Pat. No. 4,629,643, also incorporated herein by reference in the manner consistent with this disclosure and invention.

In summary, in the Curro process a sheet of a thermoplastic material is fed either from a supply roll or from a cast-embossing station onto the surface of a woven wire forming structure (or woven wire support member) which rotates about a stationary vacuum chamber. A high pressure jet of liquid, preferably at least about 800 psig, is directed at the exposed surface of a substantially smooth, flat film intermediate a pair of baffles as the web traverses the vacuum chamber. The jet of liquid causes the smooth flat sheet to plastically deform and assume the general contour of the knuckle pattern of the woven wire forming structure. In addition, because the interstices formed by the intersecting filaments are unsupported, the fluid jet causes rupture at those portions of the sheet coinciding with the interstices in the forming structure, thereby producing a planar micro-apertured film (or web). The hydroforming method results in the formation of microaberrations having volcano-shaped micro-apertures with relatively thin, irregularly shaped petals about their periphery. The outermost extremities of the petals are substantially thinner due to the elongation which occurs just prior to rupture of the film by the high pressure liquid jet.

Curro also teaches, in summary, that the “planar” micro-apertured film should be forwarded with surface micro-aberrations generally outwardly oriented about the periphery into contact with a macroscopic patterned, three-dimensional forming structure rotating about a second stationary vacuum chamber. A second stationary high pressure liquid nozzle, located intermediate stationary baffles bracketing the second vacuum chamber, provides a means for a high pressure liquid jet to apply high pressure (at least about 400 psig) across the entire width of the “planar” micro-apertured film. The high pressure liquid jet causes macroscopic expansion of the “planar” film to a three-dimensional configuration, generally resembling that of the forming structure, rotating about the second vacuum chamber.

The mesh screen and the macro-apertured screen of the Curro method are made by methods well know in the art. Both screens present a regulated pattern of apertures and therefore produce films with single regulated patterns of capillaries within each screen. In other words, the film produced on the mesh screen contains a single regulated pattern of apertures, and the film produced on the macro-patterned screen also produces a film with a single regulated pattern of apertures. Of course, if the mesh-apertured film is fed to the macro-aperturing station and subsequently it is macro-apertured, the resulting film will contain a single regulated pattern of apertures, said pattern containing both mesh-apertured and macro-apertured capillaries. Because both types of apertures are uniformly distributed over the surface of the film the final pattern is a single pattern of regulated apertures containing two types of apertures. The Curro film therefore does not contain at least two zones of regulated capillaries, it contains only one zone of regulated capillaries.

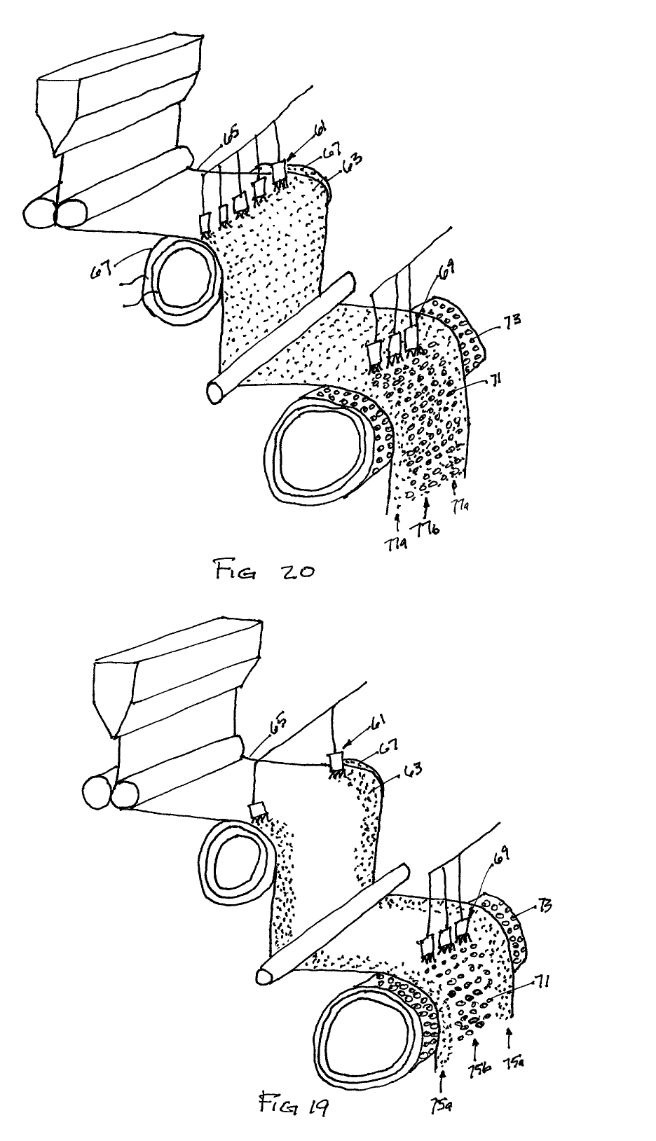

Another novel method for producing the film of the invention is an improvement of the Curro method. Referring to FIG. 19, in one embodiment of the Curro process a first high pressure liquid nozzle array 61 is used to form a first regulated pattern of passageways 63 onto a substrate film 65 corresponding to a first regulated pattern screen 67, said substrate film thereafter called “micro-apertured” film 65. Subsequently a second high pressure liquid nozzle array 69 is used to form a regulated pattern of passageways 71 onto the micro-apertured film 65 corresponding to a second regulated pattern screen 73, the resulting film having a third regulated pattern of passageways comprising both micro and macro apertures. The improved method utilizes first and second regulated pattern screens 67, 73 to produce a response surface film, the improvement consisting of aligning the high pressure liquid nozzles 61,69 in various configurations such that at least two zones 75 containing different regulated pattern of capillaries are obtained in the final film. FIG. 19 shows a schematic diagram of an exemplary apparatus and method whereby a response surface film is made containing a first zone 75a of regulated pattern capillaries comprising micro-apertures in two lanes along the edges of the film, and a second zone 75b of regulated pattern capillaries comprising macro-apertures in the center of the film (but not extending to the edges). In this method nozzles are removed or shut-off in certain sections along the pressurized liquid distribution manifold, thereby producing lanes in the film of apertured and unapertured film. The film is subsequently fed to the second forming station where nozzles are removed or shut-off in sections corresponding to the incoming film lanes. In this example nozzles are removed or shut-off in the areas corresponding to the micro-apertured film, and are operated in the areas corresponding to the unapertured film. FIG. 20 shows a schematic diagram of an exemplary apparatus and method whereby a response surface film is made containing a first zone of regulated pattern capillaries 77a comprising micro-apertures, and a second zone of regulated pattern capillaries 77b comprising both micro-apertures and macro-apertures. In this method nozzles 61 are operated along the entire width of the incoming flat film 65, thereby producing fully micro-apertured film. The film is subsequently fed to the second forming station where nozzles 69 are removed or shut-off in sections corresponding with outer regions of the web to produce film with a macro and micro apertured center lane 77b and only micro-apertured outer lanes 77a. It is obvious to one of skill in the art that the position of the macro-aperturing and micro-aperturing stations with respect to the casting die can be reversed so that the macro-apertures are made first. It is also possible to place one or the other station such that apertures are made on the opposite side of the web, thereby creating capillaries that protrude in opposite directions. Additional lanes can also be provided.

It is well known in the art to utilize a honeycomb support member inside the screen to provide support against the fluid pressure differential required to form the capillaries. Such honeycomb support members rotate with the screen. The honeycomb support member can be attached or not attached to the screen. Even when the honeycomb support member is not attached to the screen it rotates at substantially the same surface speed as the screen due to the external electromechanical drive means that cause the support member to rotate, and the film which is either or both introduced and removed from contact with the screen at a particular line speed that is the same as the surface speed of the screen. The electromechanical drive means and the line speed are matched to ensure the unattached screen and the support member rotate at substantially the same speed. Another method of producing the film of the invention is to mount various narrow screens onto the honeycomb support structure to obtain a combination screen resembling the single-piece screen shown in FIG. 17 (described in more detail below).

Usually the film of the invention is made from a polymer, such as polyethylene, e.g., low density polyethylene (LDPE), linear low density polyethylene (LLDPE) or a mixture of LDPE and LLDPE. In one embodiment the film is made from a mixture of at least about 10% by weight, or about 10% to about 50% by weight of MDPE and the remainder LDPE, LLDPE or a blend of LDPE and LLDPE. The film may also be made from a mixture of at least 10% by weight, or about 10% to about 50% by weight of HDPE and the remainder LDPE, LLDPE or a blend of LDPE and LLDPE. Each of the material formulations can include additional materials, usually in small percentages relative to the polymer, for example processing aids, colorants (e.g. whiteners), and surfactants.

The mixtures of at least about 10% by weight, or about 10% to about 50% by weight of MDPE (or HDPE) and the remainder LDPE or LLDPE or a blend of LDPE and LLDPE may also be used to make prior art films having capillaries, described above and exemplified by those of Carlucci et al., EP 0 934 733 AI.

Exemplary Screens and Screen Making Methods

FIG. 17 shows an exemplary screen 35 for use in manufacturing an apertured polymer film in accordance with the invention. The screen 35 has lanes 37 with passageways 39 of differing diameter. In the specific example of FIG. 17, outer lanes 37a and 37c have passageways 39a of smaller diameter than the passageways 39b of the center lane 37b. Accordingly, the surface area of the opening of the passageways 39a is different than the surface area of the opening of the passageways 39b. As the polymer film is extruded through the passageways 39, the different diameter passageways 39a and 39b produce capillaries of different diameter in areas of the film corresponding to the lanes 37 on the screen 35. Specifically, the screen 35 in FIG. 17 would produce an apertured film of the invention with regions of small diameter capillaries along the edges of the film, and larger diameter through the center. The resultant film would be well suited for use, for example, as a topsheet. It may be desirable to provide a screen 35 with smaller passageways 39 in a center lane (here 37b) to produce a film that is well suited for use, for example, as a backsheet. The size and number of both the lanes and the apertures can be varied to produce different configurations of films.

Another feature of screens similar to that depicted in FIG. 17, is that lanes 37 with smaller diameter passageways 39a will produce shorter capillaries in the resultant film than lanes 37 with larger diameter passageways 39b. Thus, by varying the diameter of the passageways 39 between one or more lanes, one can vary the length of the film capillaries produced by each of the lanes. Different length film capillaries can also be achieved by varying the thickness of the film, wherein a thinner film will extrude to a shorter capillary than a thicker film. Thus, by varying the thickness of the film in one or more lanes, lanes of different length capillaries can be achieved.

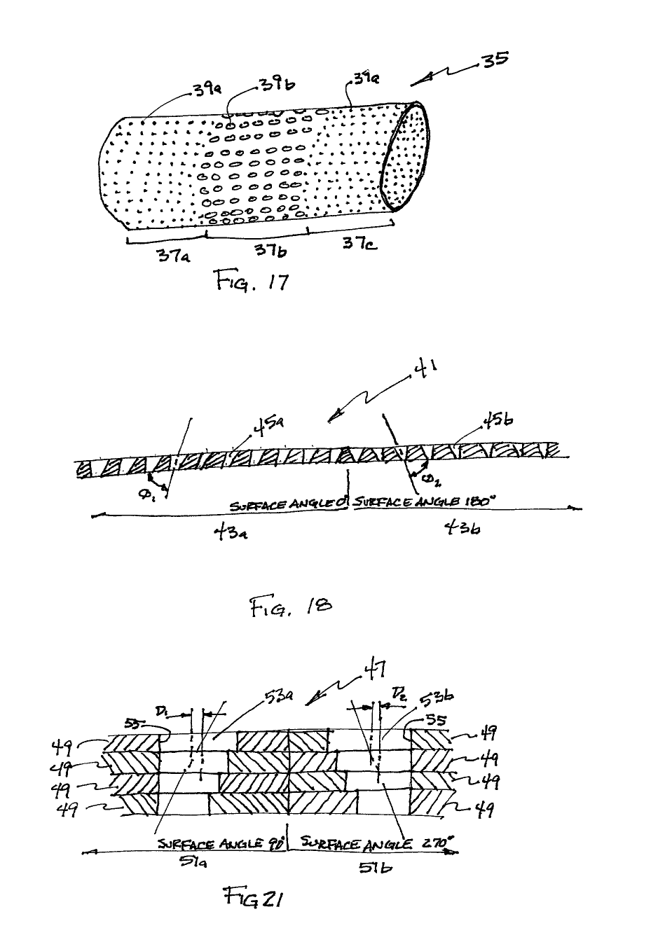

FIG. 18 shows a cross-section of an exemplary screen 41 for use in manufacturing an apertured film in accordance with the invention. The screen 41 has regions 43 of differing passageway 45 configuration. In the specific example of FIG. 18, the first region 43a has passageways 45a having a surface angle corresponding to 0 degrees and an elevation angle θ₁ measured along the centerline of the passageway. The second region 43b has passageways 45b having a surface angle corresponding to 180 degrees and the elevation angle θ₂. Elevation angles θ₁ and θ₂ may be equal, or they may be different. As the polymer film is extruded through the passageways 45, the passageways 45a produce capillaries having a 0 degree surface angle and elevation angle θ₁ in the first region 43a, and the passageways 45b produce capillaries having a 180 degree surface angle and elevation angle θ₂ in the second region 43b. Additionally, the passageways 45 depicted in FIG. 18 exhibit a taper to thereby produce corresponding capillaries of decreasing diameter. In other words, the surface area at the opening of passageways 45a (on the outer surface of the screen 41) is larger than the surface at the exit of passageways 45a (on the inner surface of the screen 41). Further, the surface area at the openings of passageways 45a can be different from the surface area at the openings of passageways 45b, and the surface area at the exits of passageways 45a can be different from the surface area at the exits of passageways 45b. The size and number of regions can be varied to produce different configuration of films. Further, the diameter, surface angle, elevation angle, and shape of the apertures can be varied to further vary the configuration of the resultant film.

The screens can be constructed by any method of screen construction known in the art. A screen similar to that of FIG. 17 (i.e. having different sized apertures) can be constructed with electroforming technique such as that described in Pruyn U.S. Pat. No. 4,383,896. Briefly, in the method disclosed in Pruyn, a screen skeleton is formed on a matrix in an electrolytic bath. In application to the present invention, the matrix would be configured to produce the desired aperture size and lane configuration. Thereafter the screen skeleton is stripped from the matrix and subjected to an electrolysis in a second electrolytic bath in the presence of at least one brightener. The second electrolytic bath is such that the growth of metal deposit on the screen skeleton is primarily perpendicular to the surface of the screen skeleton. Further detail is disclosed in the Pruyn patent, the disclosure of which is incorporated herein by reference in a manner consistent with this application and invention. An electroformed screen with response surface regions comprising a first region of pathways and a second region of pathways where the first and second regions vary only by the diameter of the pathways will typically cost no more to manufacture than a screen without response surface. Accordingly, a film with response surface regions comprising a first region of apertured capillaries and a second region of apertured capillaries where the first and second regions vary only by the diameter of the capillaries provides additional functionality at equivalent cost relative to prior art films.

Lucas, U.S. Pat. No. 4,151,240, discloses a method of making a forming screen with conical passageways. The conical passageways produce a film with conical capillaries extending perpendicular to the plane of the film. In the Lucas method, a drum (cylinder) of a sacrificial material is provided, and its outer surface is patterned with protuberances corresponding to the desired passageway size. The screen material is deposited onto the sacrificial drum and the peaks of the protuberances and portions of the screen material are removed to form openings in the deposited screen material. Alternately, the peaks of the protuberances are removed and the screen material is deposited into the valleys of the protuberances, thereby leaving openings in the deposited screen material. Thereafter the sacrificial material is selectively etched away, leaving only the screen material in the form of the final screen. Further detail is disclosed in the Lucas patent, the disclosure of which is incorporated herein by reference in a manner consistent with this application and invention.

A novel screen of the invention can be constructed in accordance with the Lucas method by shaping lanes of differing characteristics into the surface of the drum. Thus, for example, to produce a screen with larger diameter passageways in a center lane and smaller diameter passageways in the side lanes (similar to the screen of FIG. 17), the surface of the drum would be patterned with larger protuberances in the center than on the sides. The remaining steps in the Lucas method would be performed to completion, and the resulting screen would be capable of producing a film with a center lane of larger diameter apertured capillaries than the apertured capillaries of the side lanes.

Radel, U.S. Pat. No. 4,508,256, discloses a method of making a forming screen (the “PEL” method) by etching flat metal panels and stacking those panels in a manner such that the openings in the panels align to form passageways in the stack. The center axis of the passageways is disposed at a 90 degree angle from a plane parallel to the surface of the stack. In other words the passageways are aligned perpendicularly to the surface of the stack. The stacked panels are then bonded, rolled, and welded to form a cylindrical tube or screen. The passageways in the resulting screens are also aligned perpendicularly to the surface of the screens. Screens produced by this method are referred to as PEL screens. Rieker, U.S. Pat. No. 5,562,932 and U.S. Pat. No. 5,718,928 disclose an improved PEL method for producing screens capable of forming angled or curved capillaries, such as the screen of FIG. 18. The Rieker patents disclose constructing the screen from multiple panels, each panel having openings that are positioned to form passageways through the screen. By stacking the panels in a manner such that the centers of the openings in each panel are offset from the centers of the openings in the remaining panels it is possible to create passageways whose center axis is disposed at an angle different than 90 degrees from a plane parallel to the surface of the screen. In other words the passageways are aligned at a nonperpendicular angle, or slanted.

Referring to FIG. 19, a novel screen 47 of the invention (similar to the screen of FIG. 18) can be produced by arranging multiple panels 49 in such way as to produce a screen with response surface regions comprising at least a first region 51a of passageways 53a and a second region 51b of passageways 53b where the first and second regions vary by at least the slant angle of the passageways 53. FIG. 19 shows only the portion of the screen 47 at the interface of two regions 51, and though only two passageways 53 are shown, it is to be understood that there can be a plurality of passageways in each region. The slant angle consists of an elevation angle θ and a surface angle, therefore the slant angle can be varied by changing one or both of the elevation θ and surface angles. Such a screen can be produced by an improvement to the Rieker method, such improvement consisting of introducing at least two offsets when the panels are stacked. In this manner a first set of panels will be stacked with a first offset dimension (D₁) and will produce a first region of passageways characterized by a first slant angle, and a second set of panels will be stacked with a second offset dimension (D₂)and will produce a second region of passageways characterized by a second slant angle. Additionally, the passageways can be made to taper, or otherwise vary in diameter, by providing different diameter of the apertures 55 in each successive panel 49. Thus, for example, to produce a region of passageways that taper from a larger diameter (i.e. surface area) to a smaller diameter, the topmost panel in a region will have an aperture 55 of larger diameter than any of the subsequent panels 49, the subsequent panels 49 will have apertures 55 of incrementally decreasing diameter, and the bottommost panel 49 in a region will have an aperture 55 of the smallest diameter. It is important to note, also, that the shape of the passageways 53a has been made different than the shape of the passageways 53b by varying the relative position of the apertures 55 in each panel. The shape of the passageways directly affects the shape of the resultant film capillaries that are formed.

In any case, the first and second stack of panels 49 will then be bonded, and after bonding they will be positioned next to each other and welded along their edges, in this manner creating a larger composite panel. Additional bonded stacks can be positioned and welded to increase the size of the composite panel. After the composite panel is enlarged to a desirable size, the panel is rolled and a the longitudinal edges are welded to form a cylinder or screen 47.

Alternatively, a high energy beam perforation process can be used to form perpendicular or angled passageways in a cylinder to form a screen, for example, by the method described in Rose, WO 00/16726. It follows that the method and screen described in the Rose patent application can also be improved to produce novel screens and films and articles of the invention. For example, the high energy beam can be positioned at an angle relative to the surface of a rotating unapertured cylinder, and in this manner a first row of passageways can be perforated circumferentially. According to Rose the high energy beam is then slowly moved longitudinally along the length of the cylinder to add rows of passageways and to create a first zone with a regulated pattern of passageways in the cylinder of a desired first width. The angle of the high energy beam can then be changed (other high energy beam parameters such as focal distance and pulsing rate can also be changed) thereby producing a second zone with a second regulated pattern of passageways in the cylinder.

The selection of screen making processes will also enable selection of the texture of the inner surface of the screen apertures. The PEL method, due to the stacking of panels, will result in a scalloped surface. This surface can be made rougher by utilizing thicker panels, or smoother by utilizing thinner panels and increasing their number. The electroplating method (e.g. Pruyn) also affords a variety of techniques for modifying the inner surface texture of the screen apertures. For example by varying the particle size of the metals used in the plating process, by selecting different metals, etc. In the laser process (e.g. Rose) the inner surface texture is controlled by several parameters. For example if the laser perforates in a single energy pulse the surface texture of the hole will be rougher than if a narrower beam is used to first perforate and subsequently the beam is moved in circular motion to enlarge the hole. While the latter technique requires more time, it produces a much smoother surface than the single pulse perforation. The frequency and power of the laser beam also affects surface texture. For example, some laser beams melt the metal and rely upon pressurized air or other techniques to remove the molten metal. These laser methods tend to leave molten slag on the surface of the hole. Other laser beams, of different frequency and power, vaporize the metal completely leaving a smoother surface texture. Another method for controlling wall surface is by media blasting. Media blasting (e.g. sand blasting or other biasing material) not only de-glosses the surface of the screen but also de-glosses and smoothes the inner surface of the holes.

A person of skill in the art will understand that the novel methods to make a screen described above can be modified in a variety of ways, for example by changing the etched hole diameter in each zone within each panel or by etching more complex regulated patterns in the panels or by introducing more than two offset dimensions or by changing focal distances or by changing pulsing rates etc. according to the teachings of any of the preceding disclosures. The entire contents of the Rieker, Radel, and Rose patents are incorporated herein by reference in the manner consistent with this application and invention.

As discussed above, the absorbent article of the invention may comprise a topsheet and a distribution layer, which may include a film of the invention and therefore will have substantially the same properties as the backsheet which includes the film. These, and other layers, are briefly described below.

The Topsheet

The topsheets used herein may comprise a single layer or a multiplicity of layers. In a preferred embodiment the topsheet comprises a first layer which provides the user facing surface of the topsheet and a second layer between the first layer and the absorbent structure/core. In addition, another layer on the wearer facing surface of the first layer but only extending in the central zone (i.e. the area of maximum fluid discharge and areas immediately surrounding it) or in parts of the peripheral zone of the article can be desirable to provide extra softness or extra liquid handling/retaining abilities (this design is usually referred to as “hybrid topsheet”). The topsheet typically extends across the whole of the absorbent structure and can extend into and form part of or all of the preferred sideflaps, side wrapping elements or wings. The topsheet as a whole and hence each layer individually preferably need to be compliant, soft feeling, and non-irritating to the wearer's skin. It also can have elastic characteristics allowing it to be stretched in one or two directions. As used herein the topsheet refers to any layer or combination of layers whose principal function is the acquisition and transport of fluid from the wearer towards the absorbent core and containment of the absorbent core. In addition, the topsheet of the present invention should have a high vapor permeability, preferably also a high air permeability.

According to the present invention, the topsheet may be formed from any of the materials available for this purpose and known in the art, such as wovens, non-wovens, films or combinations thereof. In a preferred embodiment of the present invention, at least one of the layers of the topsheet comprises a liquid permeable apertured polymeric film of this invention. Preferably, the wearer facing and contacting layer is provided by the film material of this invention, having capillaries which are provided to facilitate liquid transport from the wearer facing surface towards the absorbent structure. The wearer facing and contacting layer may also be that disclosed, for example, in U.S. Pat. Nos. 3,929,135, 4,252,516, 4,535,020, and 5,591,510.

Absorbent Core

According to the present invention, the absorbent cores suitable for use may be selected from any of the absorbent cores or core systems known in the art. As used herein the term absorbent core refers to any material or multiple layers whose primary function is to absorb, store and distribute fluid. The absorbent core of the present invention should have a high vapor permeability, preferably also a high air permeability. The absorbent core preferably has a caliper or thickness of less than 12 mm, preferably less than 8 mm, more preferably less than 5 mm, most preferably from 4 mm to 2 mm. According to the present invention, the absorbent core can include the following components: (a) an optional primary fluid distribution layer preferably together with a secondary optional fluid distribution layer; (b) a fluid storage layer; (c) an optional fibrous (“dusting”) layer underlying the storage layer; and (d) other optional components. As discussed herein, the absorbent core may include at least one layer of the film of the invention. Such a layer may be one of the aforementioned components or it may be included in addition to such components.

Primary/Secondary Fluid Distribution Layer

One optional component of the absorbent core according to the present invention, is a primary fluid distribution layer and a secondary fluid distribution layer. The primary distribution layer typically underlies the topsheet and is in fluid communication therewith. The topsheet transfers the acquired fluid to this primary distribution layer for ultimate distribution to the storage layer. This transfer of fluid through the primary distribution layer occurs not only in the thickness, but also along the length and width directions of the absorbent product. The optional but preferred secondary distribution layer typically underlies the primary distribution layer and is in fluid communication therewith. The purpose of this secondary distribution layer is to readily acquire fluid from the primary distribution layer and transfer it rapidly to the underlying storage layer. This helps the fluid capacity of the underlying storage layer to be fully utilized. The fluid distribution layers can be comprised of any material typical for such distribution layers. At least one of the fluid distribution layers may include a film of this invention.

As discussed above, the film of the invention may be incorporated in an absorbent article having any suitable construction known in the art. One example of such an article is disclosed in Carlucci et al., published European patent application, EP 0 934 735 AI, incorporated herein by reference in the manner consistent with this application and invention. As disclosed by Carlucci et al., the absorbent article may include the following components: topsheet, absorbent core, a fluid distribution layer, which may include a primary and a secondary fluid distribution layer, a fluid storage layer, a backsheet, and an optional fibrous (“dusting”) layer. The absorbent article may also comprise other optional components, such as a reinforcing scrim positioned in the respective layers or between the respective layers. The absorbent article may also include odor control agents, such as zeolites, carbon black, silicates, EDTA, or other chelates.

The present invention is not limited to the aforementioned embodiments. Many variations of the invention can be easily envisioned by those skilled in the art without departing from the scope of the invention, as defined in the following claims.

We claim:

1. An absorbent article comprising a wearer contacting surface and a garment contacting surface, a topsheet providing a wearer contacting surface, a breathable barrier backsheet providing a garment contacting surface, and an absorbent core located in between said topsheet and said backsheet, at least one of said topsheet, said backsheet or said absorbent core including at least one layer of a macroscopically apertured film, said macroscopically apertured film comprising:
i. a first surface;
ii. a second surface that is spaced apart from said first surface; and
iii. a plurality of perforations extending through said film, each perforation forming a capillary defined by a sidewall that extends from said first surface to an aperture located in said second surface, said capillary being disposed at an elevation angle with respect to a plane that is parallel to said first surface, said capillaries being disposed at multiple elevation angles.

2. An absorbent article of claim 1 where the capillary is disposed at a surface angle.

3. An absorbent article of claim 1, wherein the sidewall extends from said first surface to an aperture located in said second surface at the apex of the capillary.

4. An absorbent article comprising a wearer contacting surface and a garment contacting surface, a topsheet providing a wearer contacting surface, a breathable barrier backsheet providing a garment contacting surface, and an absorbent core located in between said topsheet and said backsheet, at least one of said topsheet, said backsheet or said absorbent core including at least one layer of a macroscopically apertured film, said macroscopically apertured film comprising:
i. a first surface;
ii. a second surface that is spaced apart from said first surface; and
iii. a plurality of perforations extending through said film, each perforation forming a capillary defined by a sidewall that extends from said first surface to an aperture located in said second surface, said capillaries being disposed at multiple surface angles.

5. An absorbent article comprising a wearer contacting surface and a garment contacting surface, a topsheet providing a wearer contacting surface, a breathable barrier backsheet providing a garment contacting surface, and an absorbent core located in between said topsheet and said backsheet, at least one of said topsheet, said backsheet or said absorbent core including at least one layer of a macroscopically apertured film, said macroscopically apertured film comprising:
i. a first surface;
ii. a second surface that is spaced apart from said first surface; and
iii. a plurality of perforations extending through said film, each perforation forming a capillary defined by a sidewall that extends from said first surface to an aperture located in said second surface, said capillary having a water vapor permeability, said capillaries exhibiting a multiplicity of water vapor permeabilities.

6. An absorbent article of claim 5, wherein the multiplicity of water vapor permeabilities is obtained by providing at least a first capillary and a second capillary, each capillary including a first opening in the first surface, the surface area of the first opening of said first capillary being different than the surface area of the first opening of said second capillary.

7. An absorbent article of claim 5, wherein the multiplicity of water vapor permeability is obtained by providing at least a first capillary and a second capillary, each capillary, including a second opening in the second surface, the surface area of the second opening of said first capillary being different than the surface area of the second opening of said second capillary.

8. An absorbent article of claim 5, wherein the multiplicity of water vapor permeability is obtained by providing at least a first capillary and a second capillary, the shape of said first capillary being different than the shape of said second capillary.

9. An absorbent article of claim 5, wherein the multiplicity of water vapor permeability is obtained by providing at least a first capillary and a second capillary, the length of said first capillary being different than the length of said second capillary.

10. An absorbent article of claim 5, wherein the multiplicity of water vapor permeability is obtained by providing at least a first capillary and a second capillary, the surface texture of inner wall of said first capillary being different than the surface texture of inner wall of said second capillary.

11. An absorbent article comprising a wearer contacting surface and a garment contacting surface, a topsheet providing a wearer contacting surface, a breathable barrier backsheet providing a garment contacting surface, and an absorbent core located in between said topsheet and said backsheet, at least one of said topsheet, said backsheet or said absorbent core including at least one layer of a macroscopically apertured film, said macroscopically apertured film comprising:
i. a first surface;
ii. a second surface that is spaced apart from said first surface; and
iii. a plurality of perforations extending through said film, each perforation forming a capillary defined by a sidewall that extends from said first surface to an aperture located in said second surface, said capillaries exhibiting a multiplicity of levels of compression resistance.

12. An absorbent article of claim 11, wherein the multiplicity of levels of compression resistance is obtained by providing at least a first capillary comprising a first opening placed in the first surface of the film, and a second capillary comprising a first opening placed in the first surface of the film, the surface area of the first opening of said first capillary being different than the surface area of the first opening of said second capillary.

13. An absorbent article of claim 11, wherein the multiplicity of levels of compression resistance is obtained by providing at least a first capillary comprising a second opening in the second surface of the film, and a second capillary comprising a second opening in the second surface of the film, the surface area of the second opening of said first capillary being different than the surface area of the second opening of said second capillary.

14. An absorbent article of claim 11, wherein the multiplicity of levels of compression resistance is obtained by providing at least a first capillary and a second capillary, the shape of said first capillary being different than the shape of said second capillary.

15. An absorbent article of claim 11, wherein the multiplicity of levels of compression resistance is obtained by providing at least a first capillary and a second capillary, the length of said first capillary being different than the length of said second capillary.

16. An absorbent article of claim 11, wherein the multiplicity of levels of compression resistance is obtained by providing at least a first capillary and a second capillary, the distance between the first surface of the film and the second surface of the film defined by the sidewall of said first capillary being different than the distance between the first surface and the second surface defined by the sidewall of said second capillary.

17. An absorbent article of claim 11, wherein the multiplicity of levels of compression resistance is obtained by providing at least a first capillary and a second capillary, an elevation angle of said first capillary being different than an elevation angle of said second capillary.

18. An absorbent article comprising a wearer contacting surface and a garment contacting surface, a topsheet providing the wearer contacting surface; a breathable barrier backsheet providing the garment contacting surface, and an absorbent core located in between said topsheet and said backsheet, at least one of said topsheet, said backsheet or said absorbent core including at least one layer of an apertured film, said apertured film comprising:
i. a first surface;
ii. a second surface that is spaced apart from said first surface; and
iii. a plurality of apertured capillaries extending through said film, between said first surface and said second surface, each capillary defined by a side wall that extends from said first surface to said second surface, each capillary having an elevation angle, said film comprising at least a first type of apertured capillaries and a second type of apertured capillaries, the first type of apertured capillaries having a different elevation angle than the second type of apertured capillaries.

19. An absorbent article of claim 18, wherein substantially all apertured capillaries in the first type of apertured capillaries have substantially the same elevation angle.

20. An absorbent article of claim 18, wherein substantially all apertured capillaries in the second type of apertured capillaries have substantially the same elevation angle.

21. An absorbent article of claim 18, wherein said backsheet includes said film.

22. An absorbent article of claim 18, wherein said film is a macroscopically apertured polymer film.

23. An absorbent article of claim 18, which further includes a distribution layer placed between said topsheet and said absorbent core, said distribution layer being in fluid communication with said topsheet and said absorbent core, the distribution layer including at least one layer of the apertured film.

24. An absorbent article comprising a wearer contacting surface and a garment contacting surface, a topsheet providing the wearer contacting surface, a breathable barrier backsheet providing the garment contacting surface, and an absorbent core located in between said topsheet and said backsheet, at least one of said topsheet, said backsheet or said absorbent core including at least one layer of an apertured film, said apertured film comprising:
i. a first surface;
ii. a second surface that is spaced apart from said first surface; and
iii. a plurality of apertured capillaries extending through said film between said first surface and said second surface, each capillary defined by a sidewall that extends from said second surface to said first surface,
said film comprising at least a first type of apertured capillaries and a second type of apertured capillaries, the first type of apertured capillaries having a different surface angle than the second type of apertured capillaries.

25. An absorbent article of claim 24, wherein substantially all capillaries in the first type of apertured capillaries have substantially the same surface angle.

26. An absorbent article of claim 24 wherein substantially all capillaries in the second type of apertured capillaries have substantially the same surface angle.

27. An absorbent article of claim 24, wherein said backsheet includes said film.

28. An absorbent article of claim 20 wherein said film is a macroscopically expanded apertured polymer film.

29. An absorbent article of claim 24, which further includes a distribution layer placed between said topsheet and said absorbent core, said distribution layer being in fluid communication with said topsheet and said absorbent core.

30. An absorbent article comprising a wearer contacting surface and a garment contacting surface, a topsheet providing the wearer contacting surface, a breathable barrier backsheet providing the garment contacting surface, and an absorbent core located in between said topsheet and said backsheet, at least one of said topsheet, said backsheet or said absorbent core including at least one layer of an apertured film, said apertured film comprising:
i. a first surface;
ii. a second surface that is spaced apart from said first surface; and
iii. a plurality of apertured capillaries extending through said film between said first surface and said second surface, each capillary defined by a sidewall that extends from said second surface to said first surface,
said film comprising at least a first type of apertured capillaries and a second type of apertured capillaries, the first type of apertured capillaries having a different water vapor permeability than the second type of apertured capillaries.

31. An absorbent article of claim 30, wherein the different water vapor permeability is obtained by providing a different surface area of a first opening placed in the first surface of the film of said first type of apertured capillaries, than in the first opening, placed in the first surface of said film, of the second type of apertured capillaries.

32. An absorbent article of claim 30, wherein the different water vapor permeability is obtained by providing a different surface area of a second opening, placed in the second surface of said film, of said first type of apertured capillaries than in a second opening, placed in the second surface of said film, of the second type of apertured capillaries.

33. An absorbent article of claim 30, wherein the different water vapor permeability is obtained by providing the first type of apertured capillaries having a different shape than the second type of apertured capillaries.

34. An absorbent article of claim 30, wherein the different water vapor permeability is obtained by providing the first type of apertured capillaries having a different length than the second type of apertured capillaries.

35. An absorbent article of claim 30, wherein the different water vapor permeability is obtained by providing the first type of apertured capillaries having a different surface texture of their inner walls than the second type of apertured capillaries.

36. An absorbent article of claim 30 wherein the backsheet includes said film.

37. An absorbent article of claim 30 wherein said film is a macroscopically apertured polymer film.

38. An absorbent article of claim 30, which further includes a distribution layer placed between said topsheet and said absorbent core, said distribution layer being in fluid communication with said topsheet and said absorbent core, the distribution layer including at least one layer of the apertured film.

39. An absorbent article comprising a wearer contacting surface and a garment contacting surface, a topsheet providing the wearer contacting surface, a breathable barrier backsheet providing the garment contacting surface, and an absorbent core located in between said topsheet and said backsheet, at least one of said topsheet, said backsheet or said absorbent core including at least one layer of an apertured film, said apertured film comprising:
i. a first surface;
ii. a second surface that is spaced apart from said first surface; and
iii. a plurality, of apertured capillaries extending through said film between said first surface and said second surface, each capillary defined by a sidewall that extends from said second surface to said first surface,
said film comprising at least a first type of apertured capillaries and a second type of apertured capillaries, the first type of apertured capillaries having a different level of compression resistance than the second type of apertured capillaries.

40. An absorbent article of claim 39 wherein the different level of compression resistance is obtained by providing a different surface area of a first opening, placed in the first surface of said film, in the first type of apertured capillaries than in a first opening, placed in the first surface of said film, of the second type of apertured capillaries.

41. An absorbent article of claim 39 wherein the different level of compression resistance is obtained by providing a different surface area of a second opening, placed in the second surface of said film, in the first type of apertured capillaries than in a second opening, placed in the second surface of said film, in the second type of apertured capillaries.

42. An absorbent article of claim 39, wherein the different level of compression resistance is obtained by providing a different shape to said first type of capillaries than to said second type of capillaries.

43. An absorbent article of claim 39, wherein the different level of compression resistance is obtained by providing a different length to said first type of capillaries than to said second type of capillaries.

44. An absorbent article of claim 39, wherein the different level of compression resistance is obtained by providing a different distance between the first surface of the film and the second surface of the film in the first type of apertured capillaries than in the second type of apertured capillaries.

45. An absorbent article of claim 11, wherein the different level of compression resistance is obtained by providing a different elevation angle to said first type of apertured capillaries than to said second type of apertured capillaries.

46. An absorbent article of claim 39 wherein said backsheet includes said film.

47. An absorbent article of claim 39 wherein said film is a macroscopically expanded apertured polymer film.

48. An absorbent article of claim 39 which further includes a distribution layer placed between said topsheet and said absorbent core, said distribution layer being in fluid communication with said topsheet and said absorbent core, the distribution layer including at least one layer of the apertured film.

49. A macroscopically apertured film comprising:
i. a first surface;
ii. a second surface that is spaced apart from said first surface; and
iii. a plurality of perforations extending through said film, each perforation forming a capillary defined by a sidewall that extends from said first surface to an aperture located in said second surface, said capillary being disposed at an elevation angle with respect to a plane that is parallel to said first surface, said capillaries being disposed at multiple elevation angles.

50. A macroscopically apertured film comprising:
i. a first surface;
ii. a second surface that is spaced apart from said first surface; and
iii. a plurality of perforations extending through said film, each perforation forming a capillary defined by a sidewall that extends from said first surface to an aperture located in said second surface, said capillaries being disposed at multiple surface angles.

51. A macroscopically apertured film comprising:
i. a first surface;
ii. a second surface that is spaced apart from said first surface; and
iii. a plurality of perforations extending through said film, each perforation forming a capillary defined by a sidewall that extends from said first surface to an aperture located in said second surface, said capillary having a water vapor permeability, said capillaries exhibiting a multiplicity of water vapor permeabilities.

52. A film of claim 51, wherein the multiplicity of water vapor permeabilities is obtained by providing at least a first capillary and a second capillary, each capillary including a first opening in the first surface, the surface area of the first opening of said first capillary being different than the surface area of the first opening of said second capillary.

53. A film of claim 51, wherein the multiplicity of water vapor permeability is obtained by providing at least a first capillary and a second capillary, each capillary including a second opening in the second surface, the surface area of the second opening of said first capillary being different than the surface area of the second opening of said second capillary.

54. A film of claim 51, wherein the multiplicity of water vapor permeability is obtained by providing at least a first capillary and a second capillary, the shape of said first capillary being different than the shape of said second capillary.

55. A film of claim 51, wherein the multiplicity of water vapor permeability is obtained by providing at least a first capillary and a second capillary, the length of said first capillary being different than the length of said second capillary.

56. A film of claim 51, wherein the multiplicity of water vapor permeability is obtained by providing at least a first capillary and a second capillary, the surface texture of inner wall of said first capillary being different than the surface texture of inner wall of said second capillary.

57. A macroscopically apertured film comprising:
i. a first surface;
ii. a second surface that is spaced apart from said first surface; and
iii. a plurality of perforations extending through said film, each perforation forming a capillary defined by a sidewall that extends from said first surface to an aperture located in said second surface, said capillaries exhibiting a multiplicity of levels of compression resistance.

58. A film of claim 57, wherein the multiplicity of levels of compression resistance is obtained by providing at least a first capillary comprising a first opening placed in the first surface of the film, and a second capillary comprising a first opening placed in the first surface of the film, the surface area of the first opening of said first capillary being different than the surface area of the first opening of said second capillary.

59. A film of claim 57, wherein the multiplicity of levels of compression resistance is obtained by providing at least a first capillary comprising a second opening in the second surface of the film, and a second capillary comprising a second opening in the second surface of the film, the surface area of the second opening of said first capillary being different than the surface area of the second opening of said second capillary.

60. A film of claim 57, wherein the multiplicity of levels of compression resistance is obtained by providing at least a first capillary and a second capillary, the shape of said first capillary being different than the shape of said second capillary.

61. A film of claim 57, wherein the multiplicity of levels of compression resistance is obtained by providing at least a first capillary and a second capillary, the length of said first capillary being different than the length of said second capillary.

62. A film of claim 57, wherein the multiplicity of levels of compression resistance is obtained by providing at least a first capillary and a second capillary, the distance between the first surface of the film and the second surface of the film defined by the sidewall of said first capillary being different than the distance between the first surface and the second surface defined by the sidewall of said second capillary.

63. A film of claim 57, wherein the multiplicity of levels of compression resistance is obtained by providing at least a first capillary and a second capillary, an elevation angle of said first capillary being different than an elevation angle of said second capillary.

64. A film comprising:
a) a first surface;
b) a second surface which is spaced from the first surface;
c) a plurality of apertured capillaries extending between the first surface and the second surface, each capillary having an elevation angle and a surface angle;
the film comprising at least a first type and a second type of apertured capillaries, the first type of apertured capillaries having a different elevation angle than the second type of apertured capillaries.

65. A film of claim 64, wherein the first type of apertured capillaries has a different surface angle than the second type of apertured capillaries.

66. A film of claim 64, wherein the first type of apertured capillaries has a different vapor permeability than the second type of apertured capillaries.

67. A film of claim 64, wherein the first type of capillaries has a different surface area of a 1st opening than the second type of capillaries.

68. A film of claim 64, wherein the first type of capillaries has a different surface area of a 2nd opening than the second type of capillaries.

69. A film of claim 64, wherein the first type of capillaries has a different shape than the second type of capillaries.

70. A film of claim 64, wherein the first type of capillaries has a different length than the second type of capillaries.

71. A film of claim 64, wherein surface texture of an inner wall of the first type of capillaries is different than surface texture of an inner wall of A he second type of capillaries.

72. A film comprising:
a) a first surface;
b) a second surface which is spaced from the first surface;
c) a plurality of apertured capillaries extending between the first surface and the second surface, each capillary having a compression resistance;
the film comprising at least a first type of apertured capillaries and a second type of apertured capillaries, the first type of apertured capillaries having a different type of compression resistance than the second type of apertured capillaries.

73. A film of claim 72, wherein the surface area of a 1st opening of the first type of capillaries is different than the surface area of a 1st opening of the second set of capillaries.

74. A film of claim 72, wherein surface area of a 2nd opening of the first set of capillaries is different than surface area of a 2nd opening of the second set of capillaries.

75. A film of claim 72, wherein the shape of the first set of capillaries is different than the shape of the second set of capillaries.

76. A film of claim 72, wherein the length of the capillaries of the first type is different than the length of the capillaries of the second type.

77. A film of claim 72, wherein the distance between the first surface and the second surface of the first type of capillaries is different than the distance between the first surface and the second surface of the second type of capillaries.

78. A film of claim 72, wherein elevation angle of the first type of capillaries is different than elevation angle of the second type of capillaries.

79. A film of claim 72, wherein surface angle of the first type of capillaries is different than surface angle of the second type of capillaries.

80. An article of claim 1, wherein the film is made from a mixture of at least about 10% wt. of medium density polyethylene and the remainder low density polyethylene, linear low density polyethylene or a blend of low density polyethylene and linear low density polyethylene.

81. An article of claim 1, wherein the film is made from a mixture of at least about 10% wt. of high density polyethylene and the remainder low density polyethylene, linear low density polyethylene or a blend of low density polyethylene and linear low density polyethylene.

82. A film of claim 51 which is made from a mixture of at least about 10% wt. of medium density polyethylene and the remainder low density polyethylene, linear low density polyethylene or a blend of low density polyethylene and linear low density polyethylene.

83. A film of claim 51 which is made from a mixture of at least about 10% wt. of high density polyethylene and the remainder low density polyethylene, linear low density polyethylene or a blend of low density polyethylene and linear low density polyethylene.

84. A film of claim 81 whose resistance to leakage under pressure is substantially the same when the film is unwound from a roll or wound onto a roll.

85. An absorbent article comprising a wearer contacting surface and a garment contacting surface, a topsheet providing a wearer contacting surface, a breathable barrier backsheet providing a garment contacting surface, and an absorbent core located in between said topsheet and said backsheet, at least one of said topsheet, said backsheet or said absorbent core including at least one layer of a macroscopically apertured film made from a mixture of at least about 10% wt. of MDPE or HDPE and the remainder LDPE, LLDPE or a mixture of LDPE and LLDPE, said macroscopically apertured film comprising:
i. a first surface;
ii. a second surface that is spaced apart from said first surface; and
iii. a plurality of perforations extending through said film, each perforation forming a capillary defined by a sidewall that extends from said first surface to an aperture located in said second surface, said capillary being disposed at an elevation angle with respect to a plane that is parallel to said first surface.

86. An article of claim 85 wherein the film has substantially the same leakage under pressure when it is unwound from a roll as when it is wound on a roll.

87. An article of claim 83 wherein all capillaries have substantially the same elevation angles.

88. A macroscopically apertured film comprising:
i. a first surface;
ii. a second surface that is spaced apart from said first surface; and
iii. a plurality of perforations extending through said film, each perforation forming a capillary defined by a sidewall that extends from said first surface to an aperture located in said second surface, said capillary being disposed at an elevation angle with respect to a plane that is parallel to said first surface, said film made from a mixture of at least about 10% wt. of HDPE or MDPE and the remainder LDPE, LLDPE or a mixture of LDPE and LLDPE.

89. A film of claim 88 wherein all capillaries have substantially the same elevation angles.

90. A film of claim 88 whose resistance to leakage under pressure is substantially the same when the film is unwound from a roll or wound onto a roll.

91. A film of claim 88 wherein the capillaries are disposed at multiple elevation angles.

92. A film of claim 88, wherein all capillaries have substantially the same elevation angles.

93. A film of claim 88 wherein the capillaries are disposed at multiple elevation angles.

94. A screen for use in making an apertured film, comprising:
a) a first surface;
b) a second surface spaced from the first surface;
c) a plurality of passageways in a first zone of the screen, the passageways extending between the first surface and the second surface; and
d) a plurality of passageways in a second zone of the screen, the passageways extending between the first surface and the second surface, wherein the plurality of passageways in the first zone is configured to produce apertures having different characteristics than apertures produced by the plurality of passageways in the second zone.

95. A screen of claim 94, wherein the surface area of a 1st opening of at least one of the plurality of passageways in the first zone is different than the surface area of a 1st opening of at least one of the plurality of passageways in the second zone.

96. A screen of claim 94, wherein the surface area of a 2nd opening of at least one of the plurality of of passageways in the first zone is different than surface area of a 2nd opening of at least one of the plurality of passageways in the second zone.

97. A screen of claim 94, wherein the shape of at least one of the plurality of passages in the first zone is different than the shape of at least one of the plurality of passages in the second zone.

98. A screen of claim 94, wherein the elevation angle of the at least one of the plurality of passageways in the first zone is different than elevation angle of at least one of the plurality of passageways in the second zone.

99. A screen of claim 94, wherein the surface angle of at least one of the plurality of passageways in the first zone is different than surface angle of at least one of the plurality of passageways in the second zone.

100. A method of forming capillaries in an unapertured film, comprising:
passing the unapertured film over a screen;
with the screen, forming capillaries of a first slant angle in a portion of the film;
with the screen, forming capillaries of a second slant angle in a portion of the film, wherein the first slant angle is different than the second slant angle.

101. A method according to claim 100 wherein forming capillaries of a second slant angle comprises forming capillaries of a second slant angle with substantially the same elevation angle as the capillaries of the first slant angle.

102. A method according to claim 100 wherein forming capillaries of a second slant angle comprises forming capillaries of a second slant angle with substantially the same surface angle as the capillaries of the first slant angle.

103. A method according to claim 100 wherein forming capillaries of a second slant angle comprises forming capillaries of a second slant angle that are of a different shape than capillaries of the first slant angle.

104. A method according to claim 100 wherein forming capillaries of a second slant angle comprises forming capillaries of a second slant angle that have a different diameter, as measured at a surface of the film, than capillaries of the first slant angle.

105. A method of hydroforming a three-dimensional formed film, comprising:
passing an unapertured film over a first screen, the first screen having a regulated pattern of passageways therein;
directing a first fluid flow onto the unapertured film to force the film to substantially conform to the regulated pattern of the first screen in a first regulated zone;
passing the film over a second screen, the second screen having a regulated pattern of passageways therein;
directing a second fluid flow onto the film to force the film to substantially conform to the regulated pattern of the second screen in a second regulated zone; and
wherein at least a portion of the first regulated zone is separate from the second regulated zone.

106. A method in accordance with claim 105 wherein at least a portion of the first regulated zone overlaps the second regulated zone.

107. A method in accordance with claim 106 wherein the first regulated pattern produces capillaries in the film that have a smaller diameter than capillaries produced by the second regulated pattern.
